SEQUENCE LISTING (26 sequences)

SEQ ID NO: 1 - DNA, 603 bp, synthetic construct
  source 1..603: mol_type = other DNA
attcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggggtgtctgtattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcct

SEQ ID NO: 2 - DNA, 603 bp, synthetic construct
  source 1..603: mol_type = other DNA
aggactcaaggatcccagacttgtccaattatagggctccccctgtcccgccccctcccccagggatagggcgggctgggccagcccccagtcacgaggtgggctgttctccccctgccccaggcacagcagggcagagggaggaaggaggtggggcatagaggtcttttgaccaccccccacagtccccaggaagggaggacacctcgcccagtaatacagacacccccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaat

SEQ ID NO: 3 - RNA, 1234 nt, Homo sapiens
  source 1..1234: mol_type = mRNA
gggacagggggagccctataattggacaagtctgggatccttgagtcctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatcaaaaaaaaaaaaaaaaaaa

SEQ ID NO: 4 - protein, 317 aa, Homo sapiens [sequence omitted]

SEQ ID NO: 5 - RNA, 1144 nt, Homo sapiens
  source 1..1144: mol_type = mRNA
agagacgacccgacccgctagaagactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 6 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVCGRLVQYRGEVQAMLGQSTEELRVRLASHLRKLRKRLLRDPDDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 7 - RNA, 1265 nt, Homo sapiens
  source 1..1265: mol_type = mRNA
ggatggggagataagagaagaccaggagggagttaaatagggaatgggttgggggcggcttggtaaatgtgctgggattaggctgttgcagataatgcaacaaggcttggaaggctaacctgggactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatcaaaaaaaaaaaaaaaaaaa

SEQ ID NO: 8 - protein, 317 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVRGRLVQYRGEVQAMLGQSTEELRVRLASHLRKLRKRLLRDPDDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 9 - DNA, 658 bp, synthetic construct
  source 1..658: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 10 - DNA, 228 bp, synthetic construct
  source 1..228: mol_type = other DNA
gggtgtctgtattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcct

SEQ ID NO: 11 - DNA, 954 bp, synthetic construct
  source 1..954: mol_type = other DNA
atgaaggtgctgtgggccgccctgctggtgaccttcctggccggctgccaggccaaagtcgaacaggccgtcgagaccgagcccgagcccgagctgcgccagcagaccgagtggcagagcggccagcgctgggagctggccctgggccgcttctgggactacctgcgctgggtgcagaccctgagcgagcaggtgcaggaggagctgctgagcagccaggtgacccaggagctgcgcgccctgatggacgagaccatgaaagaactcaaagcttataagagcgagctggaggagcagctgacccccgtggccgaggagacccgcgcccgcctgagcaaggagctgcaggccgcccaggcccgcctgggcgccgacatggaggacgtgtgcggccgcctggtgcagtaccgcggcgaggtgcaggccatgctgggccagagcaccgaggagctgcgcgtgcgcctggccagccacctgcgcaagctgcgcaagcgcctgctgcgcgacgccgacgacctgcagaagtgcctggccgtgtaccaggccggcgcccgcgagggcgccgagcgcggcctgagcgccatccgcgagcgcctgggccccctggtggagcagggccgcgtgcgcgccgccaccgtgggcagcctggccggccagcccctgcaggagcgcgcccaggcctggggcgagcgcctgcgcgcccgcatggaggagatgggcagccgcacccgcgaccgcctggacgaggtgaaggagcaggtggccgaggtgcgcgccaagctggaggagcaggcccagcagatccgcctgcaggccgaggccttccaggcccgcctgaagagctggttcgagcccctggtggaggacatgcagcgccagtgggccggcctggtggagaaggtgcaggccgccgtgggcaccagcgccgcccccgtgcccagcgacaaccactaa

SEQ ID NO: 12 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 13 - DNA, 7351 bp, synthetic construct
  source 1..7351: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgccagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggggtgtctgtattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 14 - DNA, 7313 bp, synthetic construct
  source 1..7313: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 15 - DNA, 7047 bp, synthetic construct
  source 1..7047: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgccagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgggggtgtctgtattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 16 - DNA, 264 bp, synthetic construct
  source 1..264: mol_type = other DNA
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 17 - DNA, 21 bp, synthetic construct
  source 1..21: mol_type = other DNA
ctgcacccagcgcaggtaatc

SEQ ID NO: 18 - DNA, 21 bp, synthetic construct
  source 1..21: mol_type = other DNA
tttattaaactagggtccacc

SEQ ID NO: 19 - DNA, 54 bp, synthetic construct
  source 1..54: mol_type = other DNA
caccgattacctgcgctgggtgcagcgaactgcacccagcgcaggtaatctttt

SEQ ID NO: 20 - DNA, 150 bp, synthetic construct
  source 1..150: mol_type = other DNA
ctggaggcttgctttgggctgtatgctgtttattaaactagggtccaccttttggcctctgactgaggtggaccctagtttaataaacaggacacaaggccctttatcagcactcacatggaacaaatggccaccgtgggaggatgacaa

SEQ ID NO: 21 - DNA, 7451 bp, synthetic construct
  source 1..7451: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttactaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccaccgattacctgcgctgggtgcagcgaactgcacccagcgcaggtaatcttttttttttaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggggtgtctgtattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctgccaccatgaaggttctgtgggctgccttgcttgtgacattcctggctggatgccaggccaaagtggagcaggctgtggaaacagaacctgagcctgaactgaggcagcagacagagtggcagtctggccagagatgggaactggctttgggaaggttctgggactacctgagatgggtgcagaccctgtctgaacaggtgcaggaggagctgctgtctagccaagtgacccaagaactgagagccctgatggatgagaccatgaaggagctgaaggcctacaagtctgagctggaggagcaactcacaccagtggctgaggagacaagggctaggctgagcaaggaattgcaagctgctcaggccaggttgggagcagacatggaggatgtgtgtggcagactggtgcagtacaggggagaggttcaggctatgcttggccagagcacagaggaactgagggtgaggcttgctagccacctcaggaagctgaggaagaggctgctgagagatgctgatgacctccagaagtgcctggctgtgtatcaagctggagctagggagggagctgaaaggggactgtctgccatcagggaaagactgggacctctggttgagcagggaagagtgagagctgccacagtgggatctttggctggccaacctctgcaagaaagggctcaagcttggggagagaggcttagggccagaatggaggagatgggaagcaggacaagggacaggctggatgaggtgaaggagcaagtggctgaagtgagggccaagctggaagaacaggcccagcagattagactgcaggctgaagccttccaagccaggctgaagagctggtttgagcccttggtggaggatatgcaaaggcagtgggctggcctggttgagaaagtgcaagctgctgttggcacctctgctgctcctgtgcccagtgacaaccactgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 22 - DNA, 7640 bp, synthetic construct
  source 1..7640: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttactaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggggtgtctgtattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtctggaggcttgctttgggctgtatgctgtttattaaactagggtccaccttttggcctctgactgaggtggaccctagtttaataaacaggacacaaggccctttatcagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgaaggttctgtgggctgccttgcttgtgacattcctggctggatgccaggccaaagtggagcaggctgtggaaacagaacctgagcctgaactgaggcagcagacagagtggcagtctggccagagatgggaactggctttgggaaggttctgggactacctgagatgggtgcagaccctgtctgaacaggtgcaggaggagctgctgtctagccaagtgacccaagaactgagagccctgatggatgagaccatgaaggagctgaaggcctacaagtctgagctggaggagcaactcacaccagtggctgaggagacaagggctaggctgagcaaggaattgcaagctgctcaggccaggttgggagcagacatggaggatgtgtgtggcagactggtgcagtacaggggagaggttcaggctatgcttggccagagcacagaggaactgagggtgaggcttgctagccacctcaggaagctgaggaagaggctgctgagagatgctgatgacctccagaagtgcctggctgtgtatcaagctggagctagggagggagctgaaaggggactgtctgccatcagggaaagactgggacctctggttgagcagggaagagtgagagctgccacagtgggatctttggctggccaacctctgcaagaaagggctcaagcttggggagagaggcttagggccagaatggaggagatgggaagcaggacaagggacaggctggatgaggtgaaggagcaagtggctgaagtgagggccaagctggaagaacaggcccagcagattagactgcaggctgaagccttccaagccaggctgaagagctggtttgagcccttggtggaggatatgcaaaggcagtgggctggcctggttgagaaagtgcaagctgctgttggcacctctgctgctcctgtgcccagtgacaaccactgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 23 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 24 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKVKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNFAKSANVDFTVDNNGLYTEPRPIGTRFLTRPL

SEQ ID NO: 25 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

(19) United States
(12) Patent Application Publication
(10) Pub. No.: US 2024/0156988 A1
(43) Pub. Date: May 16, 2024

(54) SYNTHETIC NUCLEIC ACIDS INCLUDING ASTROCYTE-DIRECTED PROMOTER CONSTRUCTS AND METHODS OF USING THE SAME

(71) Applicant: Eli Lilly and Company, Indianapolis, IN (US)

(72) Inventors: Edmund Ching Schwartz, Dobbs Ferry, NY (US); Benjamin Michael Shykind, New York, NY (US)

(21) Appl. No.: 18/506,193

(22) Filed: Nov. 10, 2023

Related U.S. Application Data
(62) Division of application No. 63/383,326, filed on Nov. 11, 2022.

(51) Int. Cl. A61K 48/00; C07K 14/775; C12N 15/113; C12N 15/86

(52) U.S. Cl. CPC A61K 48/0058; A61K 48/0075; C07K 14/775; C12N 15/113; C12N 15/86; C12N 2750/14143; C12N 2750/14171; C12N 2830/008

(57) ABSTRACT

Synthetic nucleic acids are described that can be used for astrocyte-directed expression of heterologous nucleotide sequences. Also described are methods of using the same for astrocyte-directed expression of such nucleotide sequences for the treatment of neurodegenerative diseases.

### REFERENCE TO SEQUENCE LISTING SUBMITTED ELECTRONICALLY

The disclosure is being filed along with a Sequence Listing in ST.26 XML format. The Sequence Listing is provided as a file titled “30357_WO” created 31 Aug. 2023 and is 70 kilobytes (kb) in size. The Sequence Listing information in the ST.26 XML format is incorporated herein by reference in its entirety.

### TECHNICAL FIELD

The disclosure relates generally to biology and medicine, and more particularly it relates to synthetic nucleic acids that can be used as an expression control element, as well as methods of using the same for astrocyte-directed expression of heterologous nucleotide sequences, especially in treating a neurodegenerative disease.

### BACKGROUND

Alzheimer's disease (AD) is the most common form of dementia, affecting more than 5 million people in the United States alone. AD is an irreversible, progressive brain disorder characterized by the presence of abnormal protein deposits throughout the brain, which inhibit neuronal function, disrupt connections between neurons, and ultimately result in cell death. These deposits comprise plaques of amyloid-β and tangles formed by phosphorylated-tau proteins. Individuals with mild AD experience memory loss, leading to wandering, difficulty handling money, repeating questions, and personality and behavior changes. Moreover, individuals with moderate AD exhibit increased memory loss, leading to confusion and difficulty recognizing friends and family, inability to learn new things, hallucinations, delusions, and paranoia. Furthermore, individuals with severe AD cannot communicate and are completely depending on others for their care. Ultimately, protein plaques and tangles spread throughout the brain, leading to significant tissue shrinkage.

Polymorphism in the apolipoprotein E gene (APOE) is a major genetic risk determinant of late-onset AD. In the central nervous system (CNS), apolipoprotein E protein (ApoE) is mainly present in astrocytes (although some is found in microglia and stressed neurons), is the principal cholesterol carrier in the brain, and is required for cholesterol transport from astrocytes to neurons.

In addition to AD, there are a number of other neurodegenerative diseases characterized by astrocyte dysfunction including, but not limited to, gliosis associated with AD, amyotrophic lateral sclerosis (ALS) and Huntington's disease (HD).

There is a need for synthetic nucleic acids that can be used as an expression control element to drive expression principally in astrocytes, as well as methods of using the same for astrocyte-directed expression of one or more heterologous nucleotide sequences, especially in treating a neurodegenerative disease.

### BRIEF SUMMARY

To address this need, the disclosure first describes a synthetic nucleic acid that can be used as an expression control element (i.e., a promoter). In some instances, the expression control element (i.e., promoter) is for astrocyte-directed expression of one or more operably linked heterologous nucleotide sequences (i.e., a transgene and/or an inhibitory nucleic acid). In some instances, the expression control element (i.e., promoter) includes a nucleotide sequence having at least about 90% (i.e., or about 91%, about 92%, about 93%, about 94%, about 95%, about 96%, about 97%, about 98%, about 99% or about 100%) sequence identity to SEQ ID NO:1. In other instances, the expression control element (i.e., promoter) is SEQ ID NO:1.

In some instances, the synthetic nucleic acid can be an expression construct including a first nucleotide sequence having at least about 90% (i.e., or about 91%, about 92%, about 93%, about 94%, about 95%, about 96%, about 97%, about 98%, about 99% or about 100%) sequence identity to SEQ ID NO:1 operably linked to a second nucleotide sequence, where the first nucleotide sequence is an expression control element and the second nucleotide sequence encodes a transgene. In other instances, the expression construct includes a third nucleotide sequence, where the third nucleotide sequence encodes an inhibitory nucleic acid. In certain instances, the first nucleotide sequence is SEQ ID NO:1.

Alternatively, the synthetic nucleic acid can be an expression construct including a first nucleotide sequence having at least about 90% (i.e., or about 91%, about 92%, about 93%, about 94%, about 95%, about 96%, about 97%, about 98%, about 99% or about 100%) sequence identity to SEQ ID NO:1 operably linked to a second nucleotide sequence, where the first nucleotide sequence is an expression control element and the second nucleotide sequence encodes an inhibitory nucleic acid. In other instances, the expression construct includes a third nucleotide sequence, where the third nucleotide sequence encodes a transgene. In certain instances, the first nucleotide sequence is SEQ ID NO:1.

In some instances, the synthetic nucleic acid can be a vector that includes a first nucleotide sequence having at least 90% (i.e., or about 91%, about 92%, about 93%, about 94%, about 95%, about 96%, about 97%, about 98%, about 99% or about 100%) sequence identity to SEQ ID NO:1 operably linked to a second nucleotide sequence, where the first nucleotide sequence is an expression control element and the second nucleotide sequence encodes a transgene, and where the vector can be a plasmid or viral vector. In other instances, the vector includes a third nucleotide sequence, where the third nucleotide sequence encodes an inhibitory nucleic acid. In yet other instances, the vector can be a viral vector, especially a recombinant adeno-associated virus (rAAV) vector or a baculoviral vector. In certain instances, the first nucleotide sequence is SEQ ID NO:1.

Alternatively, the synthetic nucleic acid can be a vector that includes a first nucleotide sequence having at least about 90% (i.e., or about 91%, about 92%, about 93%, about 94%, about 95%, about 96%, about 97%, about 98%, about 99% or about 100%) sequence identity to SEQ ID NO:1 operably linked to a second nucleotide sequence, where the first nucleotide sequence is an expression control element and the second nucleotide sequence encodes an inhibitory nucleic acid, and where the vector is a plasmid or viral vector. In other instances, the vector includes a third nucleotide sequence, where the third nucleotide sequence encodes a transgene. In yet other instances, the vector can be a viral vector, especially a rAAV vector or a baculoviral vector. In certain instances, the first nucleotide sequence is SEQ ID NO:1.

In any of the above, the transgene can be an astrocyte-associated gene such as, for example, APOE2 or another gene associated with gliosis in AD, ALS or HD.

In any of the above, the inhibitory nucleic acid can be directed toward an astrocyte-associated gene such as, for example, APOE4 or another gene associated with gliosis in AD, ALS or HD.

Second, the disclosure describes a composition including a synthetic nucleic acid herein, such as an expression construct or vector herein. In some instances, the composition can be a rAAV that includes a capsid protein and a synthetic nucleic acid herein, including a capsid protein that can cross the blood-brain barrier (BBB). In other instances, the composition can be a host cell including a synthetic nucleic acid, such as an expression construct or vector herein. In yet other instances, the composition can be a pharmaceutical composition that includes a synthetic nucleic acid herein, such as an expression construct or vector herein and a pharmaceutically acceptable carrier.

Third, the disclosure describes a method of preferentially expressing a nucleotide sequence in an astrocyte. The method can include a step of providing an effective amount of a synthetic nucleic acid or composition herein to a cell, tissue, organ or individual.

Fourth, the disclosure describes a method of treating a neurodegenerative disease in an individual in need thereof, especially a neurodegenerative disease in which astrocyte-directed expression is desired. The method can include a step of administering to the individual an effective amount of a synthetic nucleic acid or composition herein. In some instances, the administering can be via a direct injection the CNS of the individual, which can be an intracerebroventricular (ICV) injection, an intracisterna magna (ICM) injection, an intreparenchymal injection, an intrathecal injection or a combination thereof. In certain instances, the direct injection can be convection enhanced delivery (CED). In yet other instances, the administering can be a peripheral injection. In certain instances, the peripheral injection can be via intravenous (IV) injection or subcutaneous (SC) injection.

Fifth, the disclosure describes the use of a composition including a synthetic nucleic acid herein in the manufacture of a medicament for treatment of a neurodegenerative disease, especially a neurodegenerative disease in which astrocyte-directed expression is desired.

Sixth, the disclosure describes a composition including a synthetic nucleic acid herein for use in the treatment of a neurodegenerative disease, especially a neurodegenerative disease in which astrocyte-directed expression is desired.

An advantage of the expression control element herein is that it is specific for astrocytes and thus can drive expression of a heterologous nucleotide sequence such as a transgene and/or an inhibitory nucleic acid in astrocytes to a level but not other cells in the CNS.

Another advantage of the expression control element herein is that it can be used to match endogenous expression of a target gene such as, for example, APOE, in neurodegenerative disease in which there is aberrant astrocyte-associated gene expression.

### BRIEF DESCRIPTION OF THE DRAWINGS

The advantages, effects, features, and objects other than those set forth above will become more readily apparent when consideration is given to the detailed description below. Such detailed description refers to the following drawing(s), where:

FIG. 1 shows expression of codon-optimized ApoE driven by one of three different expression control elements (i.e., one known promoter (SEQ ID NO:9 (Known Prom.; chicken beta actin (CBA)) and two potential astrocyte-specific promoters (SEQ ID NOS:1 (1^{st} Prom.) or 10 (2^{nd} Prom.)) in three different cells lines (expression relative to GAPDH control).

FIGS. 2A-2C show expression of codon-optimized ApoE driven by one of three different expression control elements (i.e., one known promoter (SEQ ID NO:9 (Known Prom.) and two potential astrocyte-specific promoters (SEQ ID NOS:1 (1^{st} Prom.) or 10 (2^{nd} Prom.)) in three different cells lines, where FIG. 2A is expression in U87 cells, where FIG. 2B is expression in HEK293 cells, and where FIG. 2C is expression in SH-SY5Y cells (fold change over known promoter).

FIGS. 3A-3B are bar graphs quantifying expression of codon-optimized ApoE2 or endogenous ApoE following transduction by rAAV having constructs driven by one of two different expression control elements (i.e., an alternative known promoter (U6; SEQ ID NO:16) or a potential astrocyte-specific promoter (1^{st} Prom; SEQ ID NO:1), where FIG. 3A shows codon-optimized ApoE2 expression (C is control (i.e., excipient), bar 1 is rAAV including the alternative promoter and 1^{st} Prom. at a multiplicity of infection (MOI) of 2×10⁴, bar 2 is rAAV including the alternative promoter and 1^{st} Prom. at a MOI of 2×10⁵, bar 3 is rAAV including the alternative promoter and the 1^{st} Prom. at a MOI of 2×10⁶, bar 4 is rAAV including the 1^{st} Prom. at a MOI of 2×10⁴, bar 5 is rAAV including the 1^{st} Prom. at a MOI of 2×10⁵ and bar 6 is rAAV including the 1^{st} Prom. at a MOI of 2×10⁶) and where FIG. 3B shows ApoE expression of the same.

FIGS. 4A-4B are images from mouse studies with promoters of SEQ ID NOS:1 or 9, where FIG. 4A shows that SEQ ID NO:9 weakly drives in vivo expression of enhanced green fluorescent protein (EGFP) in astrocytes of mouse brains and where FIG. 4B shows that SEQ ID NO:1 drives in vivo expression of EGFP in astrocytes of mouse brains (glial fibrillary acidic protein (GFAP) used as an astrocyte marker).

FIGS. 5A-5B are images from mouse studies with promoters of SEQ ID NOS:1 or 9, where FIG. 5A shows that SEQ ID NO:9 drives in vivo expression of EGFP in neurons of mouse brains and where FIG. 5B shows that SEQ ID NO:1 weakly drives in vivo expression of EGFP in neurons of mouse brains (pan neuronal marker (PNM) used as a neuronal marker).

FIGS. 6A-6B are bar graphs quantifying expression by expression control elements of SEQ ID NOS:1, 9 or 10, where FIG. 6A is GFP expression in astrocytes of the cerebral cortex and where FIG. 6B is GFP expression in astrocytes of the hippocampus.

FIGS. 7A-7B are bar graphs quantifying expression by expression control elements of SEQ ID NOS:1, 9 or 10, where FIG. 7A is GFP expression in neurons of the cerebral cortex and where FIG. 7B is GFP expression of neurons in the hippocampus.

### DETAILED DESCRIPTION

Overview

APOE is involved in the development of late-onset AD. APOE has several isoforms. One isoform, APOE2, is protective against AD; however, another isoform, APOE4, is associated with an increased risk for developing late-onset AD relative to the common isoform, APOE3. Homozygous individuals carry two copies of the APOE4 (i.e., are APOE4^{+/+}) and are at an even greater risk of developing late-onset AD as compared to heterozygous individuals who carry one copy of APOE4 and one copy of either APOE2 or APOE3 (APOE4⁺/APOE2⁺ or APOE4⁺/APOE3⁺).

Human ApoE is a 34 kDa glycoprotein having 299 amino acids after cleavage of an 18-amino acid signal peptide. The ApoE isoforms differ from one another only at positions 130 and 176 (i.e., ApoE2-Cys130 and Cys176 (see, SEQ ID NO:4); ApoE3-Cys130 and Arg176 (see, SEQ ID NO:6); and ApoE4-Arg130 and Arg176 (see, SEQ ID NO:8)).

Evidence is accumulating that ApoE influences tau pathology, tau-mediated neurodegeneration and microglial responses to AD-related pathologies. In addition, ApoE4 is either pathogenic or shows reduced efficiency in multiple brain homeostatic pathways, including lipid transport, synaptic integrity and plasticity, glucose metabolism and cerebrovascular function.

Astrocyte-directed expression of heterologous nucleotide sequences therefore is of interest in treating neurodegenerative disease such as AD, as well as diseases caused by other astrocyte-associated genes.

### Abbreviations and Definitions

Unless defined otherwise, all technical and scientific terms used herein have the same meaning as commonly understood by one of skill in the art to which the disclosure pertains. Although any methods and materials similar to or equivalent to those herein can be used in the practice or testing of the methods herein, the preferred methods and materials are herein.

Additionally, reference to an element by the indefinite article “a” or “an” does not exclude the possibility that more than one element is present, unless the context clearly requires that there be one and only one element. The indefinite article “a” or “an” thus usually means “at least one.”

Moreover, use of “including,” as well as other forms, such as “including but not limited, “include,” “includes” and “included,” is not limiting.

Certain abbreviations used herein are as follows:

“AAV” refers to adeno-associated virus; “AD” refers to Alzheimer's disease; “ALS” refers to amyotrophic lateral sclerosis; “APOE” refers to apolipoprotein E gene; “ApoE” refers to apolipoprotein E protein; “BBB” refers to blood-brain barrier; “bp” refers to base pair(s); “CBA” refers to chicken beta actin; “CED” refers to convection enhanced delivery; “DAPI” refers to 2-(4-amidinophenyl)-1H-indole-6-carboxamidine (C₁₆H₁₅N₅); “DNA” refers to deoxyribonucleic acid; “DRG” refers to dorsal root ganglion; “ds” refers to double-stranded; “EGFP” refers to enhanced green fluorescent protein; “GAPDH” refers to glyceraldehyde 3-phosphate dehydrogenase; “GFAP” refers to glial fibrillary acidic protein gene; “GFAP” refers to glial fibrillary acidic protein; “GFP” refers to green fluorescent protein; “HD” refers to Huntington's disease; “hr” refers to hour(s); “ICM” refers to intracisterna magna; “ICV” refers to intracerebroventricular; “IRES” refers to internal ribosome entry site(s); “ITR” refers to inverted terminal repeat; “IV” refers to intravenous; “kDa” refers to kilodalton(s); “min” refers to minute(s); “PNM” refers to pan neuronal marker; “qPCR” refers to quantitative reverse transcription polymerase chain reaction; “rAAV” refers to recombinant adeno-associated virus; “RNA” refers to ribonucleic acid; “RT-qPCR” refers to real-time quantitative reverse transcription polymerase chain reaction; “SC” refers to subcutaneous; “ss” refers to single-stranded; and “vg” refers to vector genome(s).

Certain definitions used herein are defined as follows:

As used herein, “about” means within a statistically meaningful range of a value or values such as, for example, a stated concentration, length, molecular weight, pH, sequence similarity, time frame, temperature, volume, etc. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, “administer,” “administering,” “administration” and the like mean providing a substance (e.g., an oligonucleotide herein or a composition herein such as a rAAV herein) to an individual in a manner that is pharmacologically useful (e.g., to treat a disease, disorder, condition or symptom in the individual).

As used herein, “astrocyte-associated gene” means a gene encoding a peptide, polypeptide or protein that is genetically, biochemically or functionally equivalent to a gene expressed predominantly in astrocytes. Exemplary astrocyte-associated genes include, but are not limited to, APOE2 and APOE4, as well as GFAP.

As used herein, “astrocyte-directed expression” means expression of a nucleotide sequence of interest encoding a peptide, polypeptide or protein predominantly in astrocytes as compared to other cells in the CNS such as, for example, neurons, including dorsal root ganglion (DRG).

As used herein, “astrocyte-specific promoter” means a promoter that drives expression of an operably linked nucleotide sequence predominantly in astrocytes as compared to other cells in the CNS such as, for example, neurons, including DRG.

As used herein, “codon-optimized” means, with respect to a nucleotide sequence such as a gene of interest such as an AD-associated gene, an alteration of codons or sequences in the gene or coding regions therein to reflect typical codon usage of a host organism (e.g., a mammal such as a human) or cell thereof without altering the polypeptide encoded by the nucleotide sequence. A codon-optimized transgene therefore is optimized for expression in a particular organism, organ, tissue or cell type, especially a mammal or mammalian organ, tissue or cell type. Alternatively, “codon-optimized” means an alteration of codons or sequences in a gene to improve protein expression as compared to a sequence that lacks the alteration by, for example, eliminating or changing sites that may be latent splice sites, stop codons, miRNA recognition sequences and the like. An entire nucleotide sequence may be codon-optimized or only one or more parts, portions or regions of a nucleotide sequence may be codon-optimized.

As used herein, “comparison window” means a contiguous and specified segment of a nucleotide sequence or amino acid sequence, where the sequence in the comparison window may include additions and/or deletions (i.e., gaps) compared to a reference sequence (which does not include the additions and/or deletions) for optimal alignment of the two sequences. Generally, the comparison window is at least 10 contiguous nucleotides/amino acids in length, and optionally can be 20, 30, 40, 50, 60, 70, 80, 90, 100 nucleotides/amino acids, or longer.

As used herein, “complementary” means a structural relationship between two nucleotides (e.g., on two opposing nucleic acids or on opposing regions of a single nucleic acid strand) that permits the two nucleotides to form base pairs (bp) with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, herein.

As used herein, “effective amount” means an amount, concentration or dose of a therapeutic agent (e.g., a nucleic acid, vector or rAAV herein), or a pharmaceutical composition thereof, upon single or multiple dose administration to an individual in need thereof, provides a desired effect in such an individual under diagnosis or treatment (i.e., may produce a clinically measurable difference in a condition of the individual). An effective amount can be readily determined by one of skill in the art by using known techniques and by observing results obtained under analogous circumstances. In determining the effective amount for an individual, a number of factors are considered, including, but not limited to, the species of mammal, its size, age and general health, the specific disease, disorder, condition or symptom involved, the degree of or involvement or the severity of the disease, disorder, condition or symptom, the response of the individual, the therapeutic agent administered, the mode of administration, the bioavailability characteristics of the preparation administered, the dose regimen selected, the use of concomitant medication, and other relevant circumstances.

As used herein, “expression construct” means a nucleotide sequence capable of replicating and expressing a nucleotide sequence of interest (e.g., a transgene or an inhibitory nucleic acid) when transformed, transfected or transduced into a target cell, tissue, organ or individual. An exemplary expression construct is a vector, such as a viral vector, especially an AAV vector or a baculovirus vector. Here, an expression construct can include at least one expression control element operably linked to the nucleotide sequence of interest such as a transgene (and/or an inhibitory nucleic acid). In this manner, the expression construct can be the expression control element, such as a promoter, in operable interaction with the transgene (and/or inhibitory nucleic acid), which is capable of directing the expression of the transgene (and/or inhibitory nucleic acid) in a cell, tissue, organ or individual, especially astrocytes.

As used herein, “expression control element” means a nucleotide sequence for a promoter, polyadenylation signal, transcription or translation termination sequence, upstream regulatory domain, origin of replication, internal ribosome entry site (IRES), enhancer and the like, which collectively provide for replication, transcription and/or translation of a desired nucleic acid (e.g., a transgene or an inhibitory nucleic acid) in a cell, tissue, organ or individual. Not all of these control sequences need always be present so long as the desired nucleotide sequence is capable of being replicated, transcribed and translated in the appropriate cell, tissue, organ or individual.

As used herein, “in combination with” means administering a therapeutic agent (e.g., nucleic acid, vector, rAAV or composition herein) either simultaneously, sequentially or in a single combined formulation with one or more additional therapeutic agents.

As used herein, “individual” means any mammal, including cats, dogs, mice, rats, and primates, especially humans. Moreover, “subject, “participant” or “patient” may be used interchangeably with “individual.”

As used herein, “individual in need thereof” means a mammal, such as a human, with a condition, disease, disorder or symptom requiring treatment or therapy, including for example, those listed herein. In particular, the preferred individual to be treated is a human.

As used herein, “inhibitory nucleic acid” means a nucleic acid molecule capable of attenuating, reducing or preventing expression of a gene or mRNA. Exemplary inhibitory nucleic acids include, but are not limited to, shRNA, siRNA, miRNA, amiRNA, etc. Here, an inhibitory nucleic acid may be a nucleotide sequence encoding for an antisense sequence to a nucleotide sequence of interest such as, for example, an AD-associated gene (e.g., a gene encoding ApoE4).

As used herein, “nucleoside” means a nucleobase-sugar combination, where the nucleobase portion is normally a heterocyclic base. The two most common classes of such heterocyclic bases are purines and pyrimidines. The sugar is normally a pentose sugar such as a ribose or a deoxyribose (e.g., 2′-deoxyribose).

As used herein, “nucleotide” means an organic molecule having a nucleoside (a nucleobase such as, for example, adenine, cytosine, guanine, thymine or uracil; and a pentose sugar such as, e.g., ribose or 2′-deoxyribose) and a phosphate group, which can serve as a monomeric unit of nucleic acid polymers such as deoxyribonucleic acid (DNA) and ribonucleic acid (RNA).

As used herein, “oligonucleotide” means a short nucleic acid molecule (e.g., less than about 100 nucleotides in length). An oligonucleotide may be single-stranded (ss) or double-stranded (ds).

As used herein, “operably linked” and the like means that the elements of an expression construct (or other nucleic acid construct) are configured so as to perform their usual function (i.e., under the influence of an expression control element). Thus, an expression control element (e.g., a promoter) operably linked to a desired nucleotide sequence (e.g., a transgene or an inhibitory nucleic acid) is capable of effecting expression of the desired nucleic acid. The control element need not be contiguous with the desired nucleotide sequence, so long as it functions to direct the expression thereof (i.e., maintain proper reading frame). Thus, for example, intervening untranslated, yet transcribed, sequence can be present between a promoter and the desired nucleotide sequence, and the promoter still can be considered “operably linked” to the desired nucleotide sequence.

As used herein, “pharmaceutically acceptable,” when referring to a material such as a carrier or diluent, means that it does not abrogate the biological activity or properties of a therapeutic agent (e.g., a nucleic acid, vector, rAAV or composition herein) and is relatively non-toxic (i.e., the material may be administered to an individual without causing undesirable biological effects or interacting in a deleterious manner with any of the components of the composition in which it is contained.

As used herein, “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a therapeutic agent within or to an individual such that it may perform its intended function. Additional ingredients that may be included in the pharmaceutical compositions used in the practice of the invention are known in the art and described, for example in Remington's Pharmaceutical Sciences, 21^{st} Edition, University of the Sciences in Philadelphia, PA (2006).

As used herein, “pharmaceutical composition” means a composition or therapeutic agent (e.g., a nucleic acid, vector, rAAV or composition herein), mixed with at least one pharmaceutically acceptable chemical component, such as, but not limited to carriers, stabilizers, diluents, dispersing agents, suspending agents, thickening agents, excipients and the like.

As used herein, “polynucleotide” means a polymer of nucleotides. Although it may comprise any type of nucleotide units, the term generally applies to nucleotide polymers of RNA or DNA. Polynucleotide is used to include ss nucleic acids, ds nucleic acids, and RNA and DNA made from nucleotide or nucleoside analogues that may be identified by their sequences, which are generally presented in the 5′ to 3′ direction (as the coding strand), where the 5′ and 3′ indicate the linkages formed between the 5′ hydroxyl group of one nucleotide and the 3′-hydroxyl group of the next nucleotide. For a coding strand presented in the 5′-3′ direction, its complement (or non-coding strand) is the strand that hybridizes to that sequence according to Watson-Crick base pairing. Thus, as used herein, the complement of a nucleic acid such as a polynucleotide is the same as the “reverse complement” and describes the nucleic acid that in its natural form, would be based paired with the nucleic acid in question.

As used herein, “recombinant adeno-associated virus,” “recombinant AAV” and “rAAV” mean viral particles comprising a rAAV vector encapsidated by AAV capsid protein.

As used herein, “recombinant adeno-associated virus vector,” “recombinant AAV vector” and “rAAV vector” mean a polynucleotide vector comprising one or more heterologous sequences (i.e., nucleic acid sequence not of an AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) that expresses AAV rep and cap gene products (i.e., AAV Rep and Cap proteins).

As used herein, “sequence identity,” in the context of two nucleotide sequences or two amino acid sequences, means that residues in the two sequences that are the same when aligned for maximum correspondence over a specified comparison window.

As used herein, “synthetic” means a nucleic acid or other molecule or compound that is artificially engineered (i.e., recombinantly produced) or that is synthesized by using a machine such as, for example, a solid phase nucleic acid synthesizer or that is otherwise not derived from a natural source that normally produces the nucleic acid or other compound (i.e., non-naturally occurring).

As used herein, “transgene” means a nucleotide sequence that is introduced into a cell and is capable of being transcribed into RNA and optionally, translated and/or expressed under appropriate conditions. The transgene confers a desired property to a cell into which it was introduced, or otherwise leads to a desired therapeutic or diagnostic outcome. Here, a transgene may be a nucleotide sequence encoding for a polypeptide of interest such as, for example, an AD-associated gene (e.g., a gene encoding ApoE2).

As used herein, “treat,” “to treat,” “treatment” or “treating” mean a process where there may be a slowing, controlling, delaying or stopping of the progression of the diseases or disorders disclosed herein, or ameliorating disease or disorder symptoms, but does not necessarily indicate a total elimination of all disease or disorder symptoms. Treatment and the like includes administration of a nucleic acid, expression construct, vector, rAAV or composition herein for treatment of a disease or disorder in an individual, particularly in a human.

As used herein, “vector” means an expression construct such as a plasmid, cosmid or phage for introducing/transferring one or more heterologous nucleotide sequences, such as an expression construct herein, to a target cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

As used herein, “viral vector” means a vector that is derived from a naturally occurring or modified virus, especially a rAAV vector or a Baculovirus vector (e.g., Autographa californica nuclear polyhedrosis (AcNPV) vector).

Compositions

Synthetic Nucleic Acids

Expression Control Elements for Use as Astrocyte-Specific Promoters: The synthetic nucleic acid can be an expression control element, such as an astrocyte-specific promoter (i.e., can be used for astrocyte-directed expression of a heterologous nucleic acid sequence such as a transgene and/or an inhibitory nucleic acid). In some instances, the synthetic nucleic acid for use as an astrocyte-specific promoter includes a nucleotide sequence having at least about 90% sequence identity to SEQ ID NO:1. Alternatively, the nucleotide sequence has at least about 90%, about 91%, about 92%, about 93%, about 94%, about 95%, about 96%, about 97%, about 98%, about 99% or about 100% sequence identity to SEQ ID NO:1. In certain instances, the nucleotide sequence is SEQ ID NO:1.

In other instances, the synthetic nucleic acid is complimentary to a nucleotide sequence having at least about 90% sequence identity to SEQ ID NO:2. Alternatively, the nucleotide sequence is complementary to a nucleotide sequence having at least about 90%, about 91%, about 92%, about 93%, about 94%, about 95%, about 96%, about 97%, about 98%, about 99% or about 100% sequence identity to SEQ ID NO:2.

The synthetic nucleic acids herein may exist on their own or may exist as part of an expression construct such as a vector or even a rAAV.

Expression Constructs: As noted above, the synthetic nucleic acid can be incorporated in an expression construct for astrocyte-directed expression of a heterologous nucleotide sequence. In some instances, the synthetic nucleic acid for use as an expression construct at least includes SEQ ID NO:1 (or a nucleotide sequence with at least about 90% to about 100% sequence identity thereto) as an expression control element and a nucleotide sequence for a transgene. In other instances, the synthetic nucleic acid for use as an expression construct at least includes SEQ ID NO:1 (or a nucleotide sequence with at least about 90% to about 100% sequence identity thereto) as an expression control element and a nucleotide sequence for an inhibitory nucleic acid. In yet other instances, the synthetic nucleic acid for use as an expression construct at least includes SEQ ID NO:1 (or a nucleotide sequence with at least about 90 to about 100% sequence identity thereto) as an expression control element, the nucleic acid sequence for the transgene and the nucleic acid sequence for the inhibitory nucleic acid. In certain instances, the expression element is SEQ ID NO:1.

In some instances, the transgene encodes an astrocyte-associated gene. In some instances, the inhibitory nucleic acid is directed toward an astrocyte-associated gene. Examples of astrocyte-associated genes include, but are not limited to, APOE2, APOE4 or another gene associated with gliosis in AD, ALS or HD.

The expression constructs herein may exist on their own or may exist as part of a vector such as a rAAV.

Vectors: As noted above, the synthetic nucleic acid or expression construct herein further can be incorporated into a vector, especially a viral vector such as an rAAV vector. A rAAV vector may comprise either the “plus strand” or the “minus strand” of the rAAV vector. In some instances, the rAAV vector is single-stranded (ss) (e.g., ss DNA or ss RNA). In other instances, the rAAV vector is double-stranded (ds) (e.g., ds DNA or ds RNA).

In other instances, the vector is a Baculovirus vector (e.g., an Autographa californica nuclear polyhedrosis (AcNPV) vector).

The vector, such as a rAAV vector, not only can include the expression control element having a nucleotide sequence of SEQ ID NO:1 (or a nucleotide sequence with at least about 90% to about 100% sequence identity thereto) and the transgene and/or inhibitory nucleic acid but also can include other expression control elements such as, for example, nucleotide sequences for at least one or more of a promoter, enhancer, transcription factor binding site, repressor binding site, intron splice site, post-transcriptional regulatory element, polyadenylation signal and combinations thereof. See, e.g., Intl. Patent Application Publication No. WO 2020/112802.

In yet other instances, the vector at least includes the expression control element having a nucleotide sequence of SEQ ID NO:1 (or a nucleotide sequence with at least about 90% to about 100% sequence identity thereto), the nucleic acid sequence for the transgene and the nucleic acid sequence for the inhibitory nucleic acid.

In some instances, the transgene encodes an astrocyte-associated gene. In some instances, the inhibitory nucleic acid is directed toward an astrocyte-associated gene. Examples of astrocyte-associated genes include, but are not limited to, APOE2, APOE4 or another gene associated with gliosis in AD, ALS or HD.

The vectors herein may exist on their own or may exist as part of a rAAV herein.

rAAV

As noted above, the synthetic nucleic acid, expression construct or vector herein can be incorporated into a rAAV. In some instances, the rAAV can have a capsid protein having a serotype selected from AAV1, AAV2, AAV3, AAV4, AAV5, AAV6, AAV7, AAV8, AAV9 and AAV10. In other instances, the rAAV may have a capsid protein from a non-human host such as, for example, a rhesus AAV capsid protein such as AAVrh10, AAVrh39, etc.

In some instances, the rAAV includes a capsid protein that is a variant of a wild-type capsid protein, where such a capsid protein variant has at least 1, 2, 3, 4, 5, 6, 7, 8, 9, 10 or more than 10 (e.g., 15, 20, 25, 50, 100, etc.) amino acid substitutions (e.g., mutations) relative to the wild-type AAV capsid protein from which it is derived.

In some embodiments, the rAAV includes a capsid protein that readily spreads through the CNS, particularly when introduced into the CSF space or directly into the brain parenchyma. In this manner, such a rAAV is capable of crossing the BBB. Examples of capsid proteins that can cross the BBB include, but are not limited to, a capsid protein having an AAV6, AAV9 or AAVrh10 serotype, as well as equivalents thereof.

With regard to AAV6 serotype (see, e.g., SEQ ID NO:23), it demonstrates tropism for the CNS, heart, liver, lung and skeletal muscle. Of particular interest herein is an AAV6 variant known as AAV6-TM (i.e., Y731F/Y705F/T492V; see, e.g., SEQ ID NO:24) or an equivalent thereto. See, e.g., Intl. Patent Application Publication Nos. WO 2008/124724, WO 2013/173512, WO 2014/193716 and WO 2016/126857; see also, Rosario et al. (2016) Mol. Ther. Methods Clin. Dev. 3:16026.

With regard to AAV9 serotype (see, e.g., SEQ ID NO:25), it displays tropism for the CNS, heart, liver, lung and skeletal muscle. See, e.g., Chen et al. (2023) Nat. Comm. 14:3345; Samaranch et al. (2012) Hum. Gen. Ther. 23:382-389 and Schuster et al. (2014) Front. Neuroanat. 8:42.

With regard to AAVrh10 serotype (see, e.g., SEQ ID NO:26), it demonstrates tropism for the CNS, heart, liver, lung and skeletal muscle. See, e.g., Hordeaux et al. (2015) Gene Therapy 22:316-324; Hoshino et al. (2019) Sci. Rep. 9:9844; and Park et al. (2017) Sci. Rep. 7:17428.

Other AAVs that can be used for CNS delivery include, but are not limited to, AAV1, AAV4 and AAV5.

Methods of producing rAAVs are described in, for example, Samulski et al. (1989) J. Viral. 63:3822-3828 and Wright (2009) Hum. Gene Ther. 20:698-706. In some instances, the rAAV can be produced in a Baculovirus vector expression system (BEVS). Production of rAAVs using BEVS are described, for example, in Urabe et al. (2002) Hum. Gene Ther. 13:1935-1943, Smith et al. (2009) Mol. Ther. 17:1888-1896, as well as U.S. Pat. Nos. 8,945,918 and 9,879,282, and Intl. Patent Application Publication Nos. WO 2017/184879 and WO 2022/082017. Alternatively, the rAAV can be produced in human embryonic kidney (e.g., HEK293) cells (see, e.g., Intl. Patent Application Publication Nos. WO 2020/210689 and WO 2022/035900). However, the rAAV can be produced using any suitable method (e.g., using recombinant rep and cap genes).

Pharmaceutical Compositions

The synthetic nucleic acids herein (i.e., an expression construct or a vector) or rAAVs herein can be formulated as a pharmaceutical composition including the synthetic nucleic acid or rAAV and a pharmaceutically acceptable carrier.

The pharmaceutical composition can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, IV administration (e.g., systemic intravenous injection), regional administration via blood and/or lymph supply, and/or direct administration to an affected site.

Generally, the most appropriate route of administration will depend upon a variety of factors including, but not limited to, the nature of the agent (e.g., its stability in the environment of its administration and/or intended target) and/or the condition of the individual (e.g., whether the subject is able to tolerate oral administration). In some instances, the synthetic nucleic acids, rAAV or pharmaceutical compositions are suitable for administration to the CNS of an individual.

Kits

In some instances, synthetic nucleic acids herein (i.e., an expression construct or a vector), rAAVs herein or even other therapeutic oligonucleotide including an expression control element herein can be included in a kit that includes the synthetic nucleic acid, rAAV or other therapeutic oligonucleotide and instructions for its use. In other instances, the kit includes the synthetic nucleic acid, rAAV or other therapeutic oligonucleotide and a package insert containing instructions for use of the kit and/or any component thereof. In yet other instances, the kit comprises, in a suitable container or other means for containing, the synthetic nucleic acid, rAAV or other therapeutic oligonucleotide, one or more controls, and various buffers, reagents, enzymes and other standard ingredients well known in the art. In some instances, the container comprises at least one vial, well, test tube, flask, bottle, syringe, or other container means, into which the synthetic nucleic acid, rAAV or other therapeutic oligonucleotide is placed, and in some instances, suitably aliquoted. In those instances where an additional component is provided, the kit includes additional containers into which this component is placed. The kits can also include a means for containing the synthetic nucleic acid, rAAV or other therapeutic oligonucleotide and any other reagent in close confinement for commercial sale. Such containers may include injection or blow-molded plastic containers into which the desired vials are retained. Containers and/or kits can include labeling with instructions for use and/or warnings.

In some instances, the kit includes the synthetic nucleic acid, rAAV or other therapeutic oligonucleotide and a pharmaceutically acceptable carrier, or a pharmaceutical composition including the synthetic nucleic acid, rAAV or other therapeutic oligonucleotide and instructions for treating or delaying progression of a neurodegenerative disease in an individual in need thereof.

In some instances, the kit includes the synthetic nucleic acid, rAAV or other therapeutic oligonucleotide and a pharmaceutically acceptable carrier or a pharmaceutical composition comprising the synthetic nucleic acid, rAAV or other therapeutic oligonucleotide and instructions for administering the synthetic nucleic acid, rAAV or other therapeutic oligonucleotide or pharmaceutical composition.

Methods

Methods of Using

The synthetic nucleic acids, rAAVs, other therapeutic oligonucleotides or pharmaceutical compositions herein may be used in a method to treat a neurodegenerative disease where such method includes at least a step of administering to an individual in need of such treatment an effect amount of a synthetic nucleic acid, rAAV, other therapeutic oligonucleotide or a pharmaceutical composition including the same.

In some instances, the synthetic nucleic acid, rAAV, other therapeutic oligonucleotide or pharmaceutical composition is administered via an IV injection or SC injection. In other instances, the synthetic nucleic acid, rAAV, other therapeutic oligonucleotide or a pharmaceutical composition is administered directly to the CNS of the individual, for example, by direct injection into the brain and/or spinal cord. Examples of direct CNS administration modalities include, but are not limited to, intracerebral injection, intraventricular injection, intracisternal injection, intraparenchymal injection, intrathecal injection, and any combination of the foregoing.

In some instances, direct CNS administration is by convection enhanced delivery (CED), which involves surgical exposing the brain and placing a small-diameter catheter directly into a target area of the brain, followed by infusion of a therapeutic agent (e.g., a synthetic nucleic acid, a rAAV, other therapeutic oligonucleotide or pharmaceutical composition herein) directly to the brain. CED is described in Debinski et al. (2009) Expert Rev. Neurother. 9:1519-1527.

In some instances, the neurodegenerative disease is an AD-associated disease. In other instances, the neurodegenerative disease is AD. In yet other instances, the individual is characterized by an APOE4 allele. The individual may be homozygous (e.g., APOE4^{+/+}) or heterozygous for APOE4 (e.g., APOE4^{+/-}). In some instances, the individual is heterozygous for APOE4 and a second APOE allele of the individual can be APOE2 or APOE3.

In some instances, the effective amount is a titer ranging from about 10⁹ Genome Copies (GC)/kg to about 10¹⁴ GC/kg. In other instances, the titer is about 10⁹ GC/kg, about 10¹⁰ GC/kg, about 10¹¹ GC/kg, about 10¹² GC/kg, about 10¹² GC/kg or about 10¹⁴ GC/kg. In yet other instances, the titer is >10¹² GC/kg by injection to the CSF space or by intraparenchymal injection.

In other instances, the effective amount is a dose ranging from about 1×10¹² vg to about 1×10¹⁵ vg or about 1×10¹³ vg to about 7×10¹⁴ vg. In other instances, the dose is about 3.5×10¹³ vg, about 7.0×10¹³ vg or about 1.4×10¹⁴ vg. In yet other instances, the dose is about 1×10¹⁴ vg, about 2.0×10¹⁴ vg, or about 4.0×10¹⁴ vg. Alternatively, the dose is about 2×10¹³ vg, about 3×10¹³ vg, about 4×10¹³ vg, about 5×10¹³ vg, about 6×10¹³ vg, about 7×10¹³ vg, about 8×10¹³ vg, about 9×10¹³ vg, about 1×10¹⁴ vg, or about 2×10¹⁴ vg. In certain instances, the dose is 7.0×10¹³ vg or 1.4×10¹⁴ vg.

In some instances, the individual is between the ages of about 1 month old to about 10 years old (e.g., about 1 month, 2 months, 3 months, 4, months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, 5 years, 6 years, 7 years, 8 years, 9 years, 10 years, or any age therebetween). In other instances, the individual is between about 10 years old to about 20 years old (e.g., about 10 years, 11 years, 12 years, 13 years, 14 years, 15 years, 16 years, 17 years, 18 years, 19 years, 20 years, or any age therebetween). In other instances, the individual is older than 20 years old (e.g., about 21 years, 22 years, 23 years, 24 years, 25 years, 26 years, 27 years, 28 years, 29 years, 30 years, or any age therebetween), older than 30 years old (e.g., about 31 years, 32 years, 33 years, 34 years, 35 years, 36 years, 37 years, 38 years, 39 years, 40 years, or any age therebetween), older than 40 years old (e.g., about 41 years, 42 years, 43 years, 44 years, 45 years, 46 years, 47 years, 48 years, 49 years, 50 years, or any age therebetween), or even older than 50 years old (e.g., about 51 years, 52 years, 53 years, 54 years, 55 years, 56 years, 57 years, 58 years, 59 years, 60 years, 70 years, 80 years, 90 years, or any age therebetween).

Uses

A rAAV or other therapeutic oligonucleotide including an expression control element herein or pharmaceutical composition including the same can be used, or adapted for use, to treat an individual (e.g., a human) having or suspected of having an AD-associated disease. As such, the rAAV or other therapeutic oligonucleotide including an expression control element herein or pharmaceutical composition including the same is provided for use, or adapted for use, to treat an individual having or suspected of having an AD-associated disease. Also provided is the use of the rAVV or other therapeutic oligonucleotide including an expression control element herein or pharmaceutical composition including the same for use, or adaptable for use, in the manufacture of a medicament or a pharmaceutical composition for treating an AD-associated disease.

### EXAMPLES

The following non-limiting examples are offered for purposes of illustration, not limitation.

In Vitro Function

### Example 1: Generating and Testing Astrocyte-Specific Expression Control Elements

Purpose: To develop expression control elements (e.g., promoters) that can drive astrocyte-specific expression of a heterologous nucleotide sequence (e.g., a transgene or an inhibitory nucleic acid).

Methods: Two putative astrocyte-specific expression control elements were generated and compared to a known promoter (SEQ ID NO:9). The first potential expression control element has a nucleotide sequence of SEQ ID NO:1, and the second potential expression control element has a nucleotide sequence of SEQ ID NO:10.

Plasmids expressing a codon-optimized human ApoE2 nucleotide sequence (SEQ ID NO:11) under the control of one of the three expression control elements were synthesized and cloned by Vigene Biosciences.

U87 (a human glioblastoma cell line), HEK293T (a human embryonic kidney cell line) and SH-SY5Y (a human neuroblastoma cell line) cell lines were transfected with these three constructs to test in vitro expression. Briefly, cells were transfected using Lipofectamine® 2000 (Invitrogen) at a 3:1 volume:mass ratio in 96 well plates, where cells were plated at a density of 30,000 cells/well. 72 hours after transfection, RNA was harvested, and gene expression was analyzed by RT-qPCR. Expression of codon-optimized ApoE was measured with glyceraldehyde 3-phosphate dehydrogenase (GAPDH) serving as a loading control.

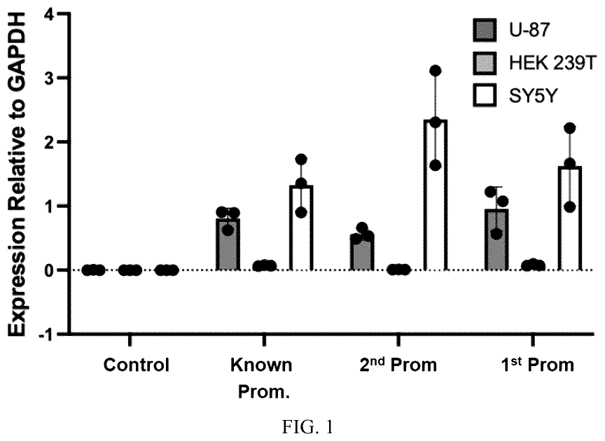

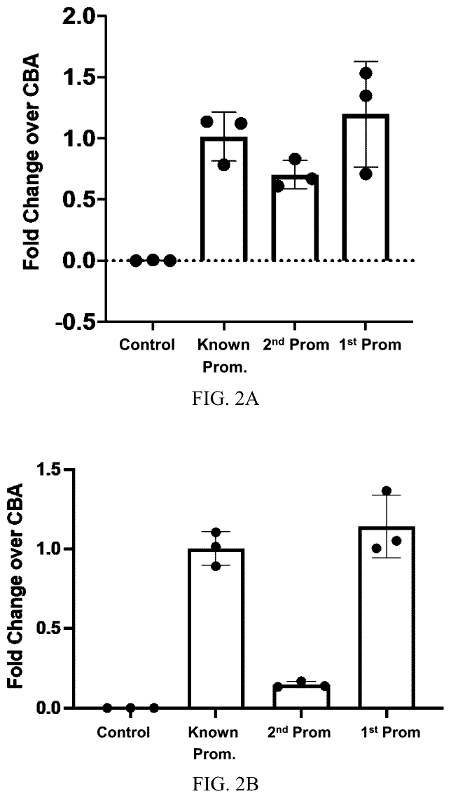

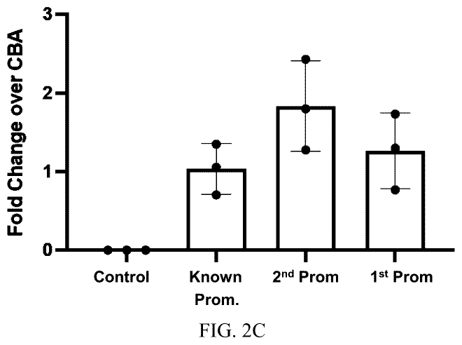

Results: In U87 cells (an astrocytoma line), all three expression control elements drove about equal expression, with the second expression control element (SEQ ID NO:10) being slightly lower (FIG. 1 and FIG. 2A). In HEK293 cells, the second expression control element (SEQ ID NO:10) had lower expression than either the known expression control element (SEQ ID NO:9) or the first expression control element (SEQ ID NO:1) (FIG. 1 and FIG. 2B). In SH5Y cells (a neuroblastoma line), the three expression control elements again drove about equal expression, with the second expression control element (SEQ ID NO:10) having potentially slightly higher expression (FIG. 1C and FIG. 2C).

### Example 2: In Vitro Studies on Astrocyte-Specific Expression of Transgenes

Purpose: To assess the ability of the astrocyte-specific expression control element of Example 1 to drive expression in an in vitro environment.

Methods: rAAV6-TM (SEQ ID NO:24) having a vector including (1) a shES01(−1) nucleotide sequence (SEQ ID NO:17) under the control of an expression control element of SEQ ID NO:16 and a codon-optimized human ApoE2 nucleotide sequence (SEQ ID NO:11) under the control of the expression control element of SEQ ID NO:1 (i.e., U6_APOE_shES01(−1)_1^{st} Prom._ApoE2CpG10; SEQ ID NO:21) or (2) a ES07c_sh nucleotide sequence (SEQ ID NO:18) in an eSibr backbone (SEQ ID NO:20; see also, Fowler et al. (2016) Nuc. Acids Res. 44:e48) and a codon-optimized human ApoE2 nucleotide sequence (SEQ ID NO:11) both under the control of the expression control element of SEQ ID NO:1 (i.e., 1^{st} Prom._eSibr_APOE ESO7c_sh_ApoE2CpG10; SEQ ID NO:22) were generated by Virovek, Inc.

U87 cells were transduced with the rAAV at a MOI of 2×10⁴ to 2×10⁶. Briefly, cells were plated in 96-well plates and treated with starvation media (2% FBS) containing rAAV and Hoechst reagent. After 2 hrs at 37° C., the starvation media was replaced with complete media (10% FBS) containing Hoechst reagent. After ˜72 hrs, RNA was harvested, and gene expression was analyzed by RT-QPCR. Expression of the codon-optimized ApoE2 and endogenous ApoE was measured with GAPDH serving as a loading control.

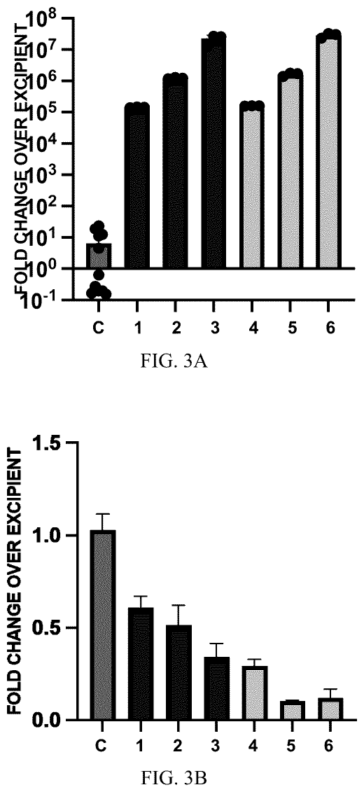

Results: In U87 cells, ApoE2 expression was comparable between the two rAAV, which appeared to be dose dependent. Differences, however, were observed in ApoE inhibition. Both rAAV demonstrated knock-down of endogenous ApoE. The sequence shES01(−1) resulted in ˜70% reduction in endogenous ApoE when driven by the U6 promoter, while ES07c resulted in ˜90% reduction in endogenous ApoE when driven by 1^{st} Prom. (SEQ ID NO:1; see, FIGS. 3A-3B).

### In Vivo Function

### Example 3: In Vivo Studies on Astrocyte-Specific Expression of Enhanced GFP

Purpose: To assess the ability of the astrocyte-specific expression control element of Example 1 to drive expression in an in vivo environment.

Methods: rAAV9 expressing enhanced GFP (EGFP; SEQ ID NO:12) under the control of the three expression control elements were generated by Virovek, Inc (expression control elements of SEQ ID NOS:1 9 and 10) or Prevail Therapeutics (expression control element of SEQ ID:9). The rAAV had the following nucleotide sequences: SEQ ID NOS:13, SEQ ID NO:14 and SEQ ID NO:15. rAAV was administered by unilateral ICV injection (4.84×10¹⁰ vg in 4 uL per animal; 6 animals for each of the 3 expression control elements) to neonatal (P2) C57BL/6 mice at Psychogenics, Inc. Animals were euthanized 4 weeks post-injection, and tissue was collected for molecular biology and imaging analysis.

Brain, spinal cord and liver were mounted for imaging, and GFP fluorescence was visualized with a DAPI nuclear counterstain.

Following the studies, DNA and mRNA were extracted from cortical, spinal cord and liver samples and analyzed by qPCR and RT-qPCR respectively to determine viral biodistribution and GFP mRNA expression.

Results: AAV using the known expression control element of SEQ ID NO:9 led to extensive expression throughout the brain almost exclusively in neurons, while AAV using the first expression control element of SEQ ID NO:1 led to about equal expression; however, the expression was localized largely in astrocytes (confirmed with preliminary GFAP counterstain). In contrast, AAV using the second expression control element of SEQ ID NO:10 led to low levels of expression in only a few cells, which were mostly neurons.

Expression in the spinal cord is qualitatively different between the first expression control element of SEQ ID NO:1 and the known expression control element of SEQ ID NO:9, while still being overall about equal.

Expression in liver appears almost identical between the first expression control element of SEQ ID NO:1 and the known expression control element of SEQ ID NO:9.

With regard to the second expression control element of SEQ ID NO:10, almost no fluorescence could be detected in either the liver or the spinal cord.

There were no significant differences between the AAV in biodistribution across all tissues (i.e., brain, liver and spinal cord). In fact, EGFP mRNA levels were similar or identical between the known expression control element of SEQ ID NO:9 and the first expression control element of SEQ ID NO:1 but was up to 10-fold lower with the second expression control element of SEQ ID NO:10.

In view of the similar biodistribution and overall expression levels between the known expression control element of SEQ ID NO:9 and the first expression control element of SEQ ID NO:1 and the same viral capsid being used for all constructs, the differences in expression pattern may be attributed to the differences in effectiveness of the expression control elements to function as a promoter. Expression from the first expression control element of SEQ ID NO:1 was predominantly in astrocytes, which was not achieved with the second expression control element of SEQ ID NO:10. Surprisingly, these in vivo data could not be predicted from the in vitro data in which expression in a neuron-like cell line (SH-SY5Y) and an astrocyte-like cell line (U87) was similar across all three expression control elements.

### Example 4: In Vivo Studies on Astrocyte-Specific Expression of GFP

Purpose: To further assess the ability of the astrocyte-specific expression control element of Example 1 in an in vivo environment.

Methods: In vivo validation was performed at PsychoGenics, Inc. (Paramus, NJ). Briefly, rAAV9 encoding EGFP (as in Example 3) under the control of an expression construct having SEQ ID NO:1, 9 or 10 was administered by ICV injection in C57BL/6 mice of mixed gender at P2. rAAV was administered at a concentration of 1.21×10¹³ vg/mL in 4 μL volume for a total dose of 4.84×10¹⁰ vg/animal. Thirty days after ICV administration, animals were euthanized and tissues were collected and either fixed for immunohistochemistry or flash frozen for molecular biology analysis. The brain, spinal cord and liver were stained with antibodies against GFAP to identify astrocytes and PNM to identify neurons and were co-imaged with native GFP fluorescence to determine cell expression. The cortex, spinal cord and liver were also analyzed for biodistribution and GFP mRNA expression by qPCR at Prevail Therapeutics (New York, NY).

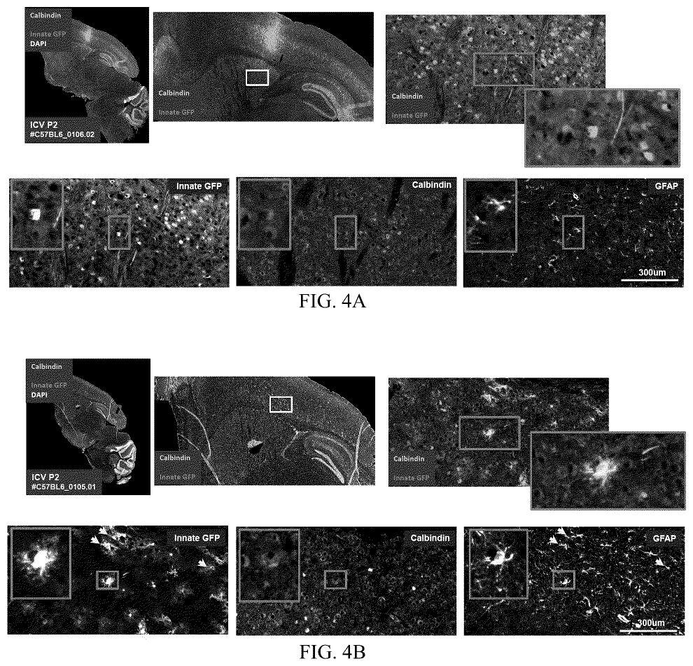

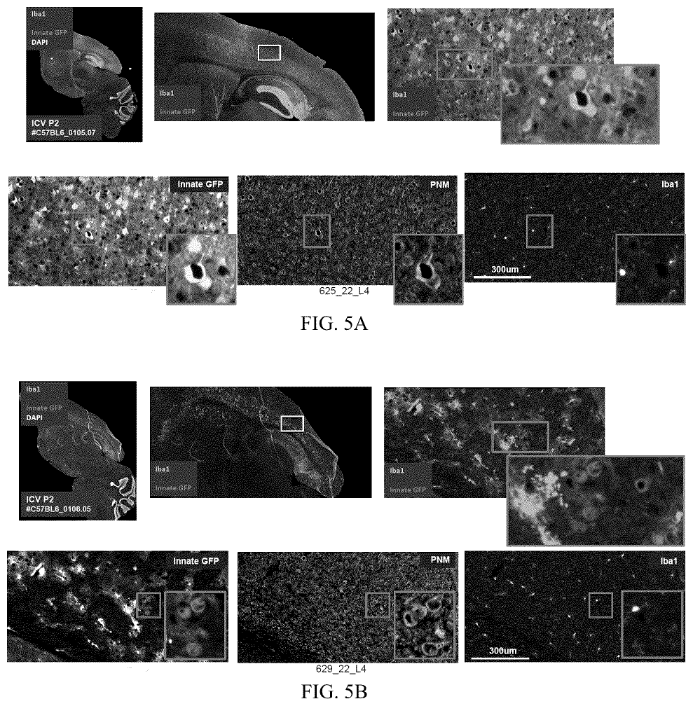

Results: The expression construct under the control of SEQ ID NO:9 weakly drives in vivo expression of EGFP in astrocytes of mouse brains (FIG. 3A). In contrast, the expression construct under the control of SEQ ID NO:1 drives in vivo expression of EGFP in astrocytes of mouse brains (FIG. 3B). However, the expression construct under the control of SEQ ID NO:9 drives in vivo expression of EGFP in neurons of mouse brains (FIG. 4A). In contrast, the expression construct under the control of SEQ ID NO:1 weakly drives in vivo expression of EGFP in neurons of mouse brains (FIG. 4B).

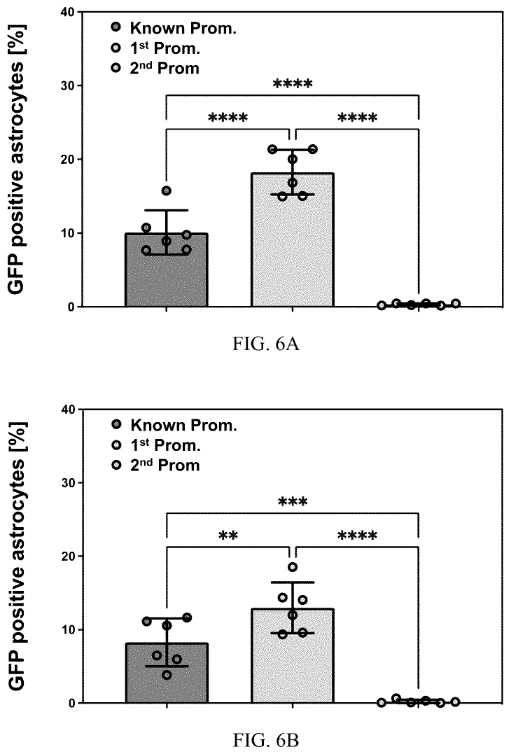

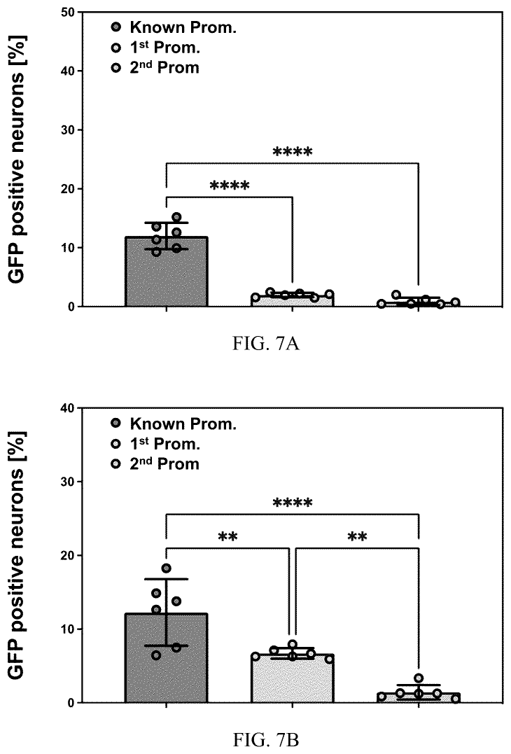

When quantified, the expression construct under the control of SEQ ID NO:1 drove expression in a larger fraction of astrocytes and a smaller fraction of neurons than expression constructs under the control of SEQ NO:9 in both the hippocampus and cortical regions (FIGS. 6A-B and 7A-B). Moreover, the expression construct under the control of SEQ ID NO:2 drove expression in only a small fraction of cells in both the hippocampus and cortical regions (FIGS. 6A-B and 7A-B).

### SEQUENCE LISTING

The following nucleotide and/or amino acid sequences are referred to in the disclosure above and are provided below for reference.

Synthetic nucleic acid 1 (603 nt)
SEQ ID NO: 1
attcggtacctagttattaatagtaatcaattacggggtcattagttcatagcccatatatggagttccgcgttacataacttacggtaaatg
gcccgcctggctgaccgcccaacgacccccgcccattgacgtcaataatgacgtatgttcccatagtaacgccaatagggactttccat
tgacgtcaatgggtggagtatttacggtaaactgcccacttggcagtacatcaagtgtatcatatgccaagtacgccccctattgacgtca
atgacggtaaatggcccgcctggcattatgcccagtacatgaccttatgggactttcctacttggcagtacatctacgtattagtcatcgct
attaccatgggggtgtctgtattactgggcgaggtgtcctcccttcctggggactgtggggggtggtcaaaagacctctatgccccacct
ccttcctccctctgccctgctgtgcctggggcagggggagaacagcccacctcgtgactgggggctggcccagcccgccctatccct
gggggagggggcgggacagggggagccctataattggacaagtctgggatccttgagtcct
Synthetic nucleic acid 2 (603 nt; complimentary sequence to SEQ ID NO: 1)
SEQ ID NO: 2
aggactcaaggatcccagacttgtccaattatagggctccccctgtcccgccccctcccccagggataggggggctgggccagcc
cccagtcacgaggtgggctgttctccccctgccccaggcacagcagggcagagggaggaaggaggtggggcatagaggtcttttg
accaccccccacagtccccaggaagggaggacacctcgcccagtaatacagacacccccatggtaatagcgatgactaatacgtag
atgtactgccaagtaggaaagtcccataaggtcatgtactgggcataatgccaggcgggccatttaccgtcattgacgtcaatagggg
gcgtacttggcatatgatacacttgatgtactgccaagtgggcagtttaccgtaaatactccacccattgacgtcaatggaaagtccctatt
ggcgttactatgggaacatacgtcattattgacgtcaatgggcgggggtcgttgggcggtcagccaggcgggccatttaccgtaagtta
tgtaacgcggaactccatatatgggctatgaactaatgaccccgtaattgattactattaataactaggtaccgaat
human APOE2 mRNA (1234 nt; NCBI Ref. Seq. No. NM_000041.3)
SEQ ID NO: 3
gggacagggggagccctataattggacaagtctgggatccttgagtcctactcagccccagcggaggtgaaggacgtccttccccag
gagccgactggccaatcacaggcaggaagatgaaggttctgtgggctgcgttgctggtcacattcctggcaggatgccaggccaag
gtggagcaagcggtggagacagagccggagcccgagctgcgccagcagaccgagtggcagagcggccagcgctgggaactgg
cactgggtcgcttttgggattacctgcgctgggtgcagacactgtctgagcaggtgcaggaggagctgctcagctcccaggtcaccca
ggaactgagggcgctgatggacgagaccatgaaggagttgaaggcctacaaatcggaactggaggaacaactgaccccggtggc
ggaggagacgcgggcacggctgtccaaggagctgcaggcggcgcaggcccggctgggcgcggacatggaggacgtgtgcggc
cgcctggtgcagtaccgcggcgaggtgcaggccatgctcggccagagcaccgaggagctgcgggtgcgcctcgcctcccacctg
cgcaagctgcgtaagcggctcctccgcgatgccgatgacctgcagaagcgcctggcagtgtaccaggccggggcccgcgagggc
gccgagcgcggcctcagcgccatccgcgagcgcctggggcccctggtggaacagggccgcgtgcgggccgccactgtgggctc
cctggccggccagccgctacaggagcgggcccaggcctggggcgagcggctgcgcgcgcggatggaggagatgggcagccgg
acccgcgaccgcctggacgaggtgaaggagcaggtggcggaggtgcgcgccaagctggaggagcaggcccagcagatacgcct
gcaggccgaggccttccaggcccgcctcaagagctggttcgagcccctggtggaagacatgcagcgccagtgggccgggctggtg
gagaaggtgcaggctgccgtgggcaccagcgccgcccctgtgcccagcgacaatcactgaacgccgaagcctgcagccatgcga
ccccacgccaccccgtgcctcctgcctccgcgcagcctgcagcgggagaccctgtccccgccccagccgtcctcctggggtggac
cctagtttaataaagattcaccaagtttcacgcatcaaaaaaaaaaaaaaaaaaa
human ApoE2 protein (317 aa)
SEQ ID NO: 4
MKVLWAALLVTFLAGCQAKVEQAVETEPEPELRQQTEWQSGQRWELALGRFWDY
LRWVQTLSEQVQEELLSSQVTQELRALMDETMKELKAYKSELEEQLTPVAEETRAR
LSKELQAAQARLGADMEDVCGRLVQYRGEVQAMLGQSTEELRVRLASHLRKLRKR
LLRDADDLQKCLAVYQAGAREGAERGLSAIRERLGPLVEQGRVRAATVGSLAGQPL
QERAQAWGERLRARMEEMGSRTRDRLDEVKEQVAEVRAKLEEQAQQIRLQAEAFQ
ARLKSWFEPLVEDMQRQWAGLVEKVQAAVGTSAAPVPSDNH
human APOE3 mRNA (1144 nt; NCBI Ref. Seq. No. NM_001302689.2)
SEQ ID NO: 5
agagacgacccgacccgctagaagactggccaatcacaggcaggaagatgaaggttctgtgggctgcgttgctggtcacattcctgg
caggatgccaggccaaggtggagcaagcggtggagacagagccggagcccgagctgcgccagcagaccgagtggcagagcgg
ccagcgctgggaactggcactgggtcgcttttgggattacctgcgctgggtgcagacactgtctgagcaggtgcaggaggagctgct
cagctcccaggtcacccaggaactgagggcgctgatggacgagaccatgaaggagttgaaggcctacaaatcggaactggaggaa
caactgaccccggtggcggaggagacgcgggcacggctgtccaaggagctgcaggcggcgcaggcccggctgggcgcggaca
tggaggacgtgtgcggccgcctggtgcagtaccgcggcgaggtgcaggccatgctcggccagagcaccgaggagctgcgggtgc
gcctcgcctcccacctgcgcaagctgcgtaagcggctcctccgcgatgccgatgacctgcagaagcgcctggcagtgtaccaggcc
ggggcccgcgagggcgccgagcgcggcctcagcgccatccgcgagcgcctggggcccctggtggaacagggccgcgtgcggg
ccgccactgtgggctccctggccggccagccgctacaggagcgggcccaggcctggggcgagcggctgcgcgcgcggatggag
gagatgggcagccggacccgcgaccgcctggacgaggtgaaggagcaggtggcggaggtgcgcgccaagctggaggagcagg
cccagcagatacgcctgcaggccgaggccttccaggcccgcctcaagagctggttcgagcccctggtggaagacatgcagcgcca
gtgggccgggctggtggagaaggtgcaggctgccgtgggcaccagcgccgcccctgtgcccagcgacaatcactgaacgccgaa
gcctgcagccatgcgaccccacgccaccccgtgcctcctgcctccgcgcagcctgcagcgggagaccctgtccccgccccagccg
tcctcctggggtggaccctagtttaataaagattcaccaagtttcacgca
human ApoE3 protein (317 aa)
SEQ ID NO: 6
MKVLWAALLVTFLAGCQAKVEQAVETEPEPELRQQTEWQSGQRWELALGRFWDY
LRWVQTLSEQVQEELLSSQVTQELRALMDETMKELKAYKSELEEQLTPVAEETRAR
LSKELQTAQARLGADMEDVCGRLVQYRGEVQAMLGQSTEELRVRLASHLRKLRKR
LLRDPDDLQKRLAVYQAGAREGAERGLSAIRERLGPLVEQGRVRAATVGSLAGQPL
QERAQAWGERLRARMEEMGSRTRDRLDEVKEQVAEVRAKLEEQAQQIRLQAEAFQ
ARLKSWFEPLVEDMQRQWAGLVEKVQAAVGTSAAPVPSDNH
human APOE4 mRNA (1265 nt; NCBI Ref. Seq. No. NM_001302690.1)
SEQ ID NO: 7
ggatggggagataagagaagaccaggagggagttaaatagggaatgggttgggggcggcttggtaaatgtgctgggattaggctgt
tgcagataatgcaacaaggcttggaaggctaacctgggactggccaatcacaggcaggaagatgaaggttctgtgggctgcgttgct
ggtcacattcctggcaggatgccaggccaaggtggagcaagcggtggagacagagccggagcccgagctgcgccagcagaccg
agtggcagagcggccagcgctgggaactggcactgggtcgcttttgggattacctgcgctgggtgcagacactgtctgagcaggtgc
aggaggagctgctcagctcccaggtcacccaggaactgagggcgctgatggacgagaccatgaaggagttgaaggcctacaaatc
ggaactggaggaacaactgaccccggtggcggaggagacgcgggcacggctgtccaaggagctgcaggcggcgcaggcccgg
ctgggcgcggacatggaggacgtgtgcggccgcctggtgcagtaccgcggcgaggtgcaggccatgctcggccagagcaccga
ggagctgcgggtgcgcctcgcctcccacctgcgcaagctgcgtaagcggctcctccgcgatgccgatgacctgcagaagcgcctg
gcagtgtaccaggccggggcccgcgagggcgccgagcgcggcctcagcgccatccgcgagcgcctggggcccctggtggaaca
gggccgcgtgcgggccgccactgtgggctccctggccggccagccgctacaggagcgggcccaggcctggggcgagcggctgc
gcgcgcggatggaggagatgggcagccggacccgcgaccgcctggacgaggtgaaggagcaggtggcggaggtgcgcgccaa
gctggaggagcaggcccagcagatacgcctgcaggccgaggccttccaggcccgcctcaagagctggttcgagcccctggtggaa
gacatgcagcgccagtgggccgggctggtggagaaggtgcaggctgccgtgggcaccagcgccgcccctgtgcccagcgacaat
cactgaacgccgaagcctgcagccatgcgaccccacgccaccccgtgcctcctgcctccgcgcagcctgcagcgggagaccctgt
ccccgccccagccgtcctcctggggtggaccctagtttaataaagattcaccaagtttcacgcatcaaaaaaaaaaaaaaaaaaa
human ApoE4 protein (317 aa)
SEQ ID NO: 8
MKVLWAALLVTFLAGCQAKVEQAVETEPEPELRQQTEWQSGQRWELALGRFWDY
LRWVQTLSEQVQEELLSSQVTQELRALMDETMKELKAYKSELEEQLTPVAEETRAR
LSKELQTAQARLGADMEDVRGRLVQYRGEVQAMLGQSTEELRVRLASHLRKLRKR
LLRDPDDLQKRLAVYQAGAREGAERGLSAIRERLGPLVEQGRVRAATVGSLAGQPL
QERAQAWGERLRARMEEMGSRTRDRLDEVKEQVAEVRAKLEEQAQQIRLQAEAFQ
ARLKSWFEPLVEDMQRQWAGLVEKVQAAVGTSAAPVPSDNH
Synthetic nucleic acid 3 (658 nt)
SEQ ID NO: 9
gacattgattattgactagttattaatagtaatcaattacggggtcattagttcatagcccatatatggagttccgcgttacataacttacggt
aaatggcccgcctggctgaccgcccaacgacccccgcccattgacgtcaataatgacgtatgttcccatagtaacgccaatagggact
ttccattgacgtcaatgggtggactatttacggtaaactgcccacttggcagtacatcaagtgtatcatatgccaagtacgccccctattga
cgtcaatgacggtaaatggcccgcctggcattatgcccagtacatgaccttatgggactttcctacttggcagtacatctacgtattagtc
atcgctattaccatgtcgaggtgagccccacgttctgcttcactctccccatctcccccccctccccacccccaattttgtatttatttatttttt
aattattttgtgcagcgatgggggcggggggggggggggggcgcgcgccaggcggggcggggcggggcgaggggggggcg
gggcgaggcggagaggtgcggcggcagccaatcagagcggcgcgctccgaaagtttccttttatggcgaggcggcggcggcggc
ggccctataaaaagcgaagcgcgcggcgggcg
Synthetic nucleic acid 4 (228 nt)
SEQ ID NO: 10
gggtgtctgtattactgggcgaggtgtcctcccttcctggggactgtggggggtggtcaaaagacctctatgccccacctccttcctccc
tctgccctgctgtgcctggggcagggggagaacagcccacctcgtgactgggggctggcccagcccgccctatccctgggggagg
ggggggacagggggagccctataattggacaagtctgggatccttgagtcct
Synthetic nucleic acid 5 (954 nt)
SEQ ID NO: 11
atgaaggtgctgtgggccgccctgctggtgaccttcctggccggctgccaggccaaagtcgaacaggccgtcgagaccgagcccg
agcccgagctgcgccagcagaccgagtggcagagcggccagcgctgggagctggccctgggccgcttctgggactacctgcgct
gggtgcagaccctgagcgagcaggtgcaggaggagctgctgagcagccaggtgacccaggagctgcgcgccctgatggacgag
accatgaaagaactcaaagcttataagagcgagctggaggagcagctgacccccgtggccgaggagacccgcgcccgcctgagc
aaggagctgcaggccgcccaggcccgcctgggcgccgacatggaggacgtgtgcggccgcctggtgcagtaccgcggcgaggt
gcaggccatgctgggccagagcaccgaggagctgcgcgtgcgcctggccagccacctgcgcaagctgcgcaagcgcctgctgcg
cgacgccgacgacctgcagaagtgcctggccgtgtaccaggccggcgcccgcgagggcgccgagcgcggcctgagcgccatcc
gcgagcgcctgggccccctggtggagcagggccgcgtgcgcgccgccaccgtgggcagcctggccggccagcccctgcagga
gcgcgcccaggcctggggcgagcgcctgcgcgcccgcatggaggagatgggcagccgcacccgcgaccgcctggacgaggtg
aaggagcaggtggccgaggtgcgcgccaagctggaggagcaggcccagcagatccgcctgcaggccgaggccttccaggcccg
cctgaagagctggttcgagcccctggtggaggacatgcagcgccagtgggccggcctggtggagaaggtgcaggccgccgtggg
caccagcgccgcccccgtgcccagcgacaaccactaa
Enhanced green fluorescent protein (720 nt)
SEQ ID NO: 12
Atggtgagcaagggcgaggagctgttcaccggggtggtgcccatcctggtcgagctggacggcgacgtaaacggccacaagttca
gcgtgtccggcgagggcgagggcgatgccacctacggcaagctgaccctgaagttcatctgcaccaccggcaagctgcccgtgcc
ctggcccaccctcgtgaccaccctgacctacggcgtgcagtgcttcagccgctaccccgaccacatgaagcagcacgacttcttcaa
gtccgccatgcccgaaggctacgtccaggagcgcaccatcttcttcaaggacgacggcaactacaagacccgcgccgaggtgaagt
tcgagggcgacaccctggtgaaccgcatcgagctgaagggcatcgacttcaaggaggacggcaacatcctggggcacaagctgga
gtacaactacaacagccacaacgtctatatcatggccgacaagcagaagaacggcatcaaggtgaacttcaagatccgccacaacat
cgaggacggcagcgtgcagctcgccgaccactaccagcagaacacccccatcggcgacggccccgtgctgctgcccgacaacca
ctacctgagcacccagtccgccctgagcaaagaccccaacgagaagcgcgatcacatggtcctgctggagttcgtgaccgccgccg
ggatcactctcggcatggacgagctgtacaagtaa
CAPO-GFP AAV expression construct/vector (7351 nt)
SEQ ID NO: 13
cattcgccattcaggctgcaaataagcgttgatattcagtcaattacaaacattaataacgaagagatgacagaaaaattttcattctgtga
cagagaaaaagtagccgaagatgacggtttgtcacatggagttggcaggatgtttgattaaaaacataacaggaagaaaaatgccccg
ctgtgggcggacaaaatagttgggaactgggaggggtggaaatggagtttttaaggattatttagggaagagtgacaaaatagatggg
aactgggtgtagcgtcgtaagctaatacgaaaattaaaaatgacaaaatagtttggaactagatttcacttatctggttcggatctcctaga
gcttacagcttcctgcaggcagctgcgcgctcgctcgctcactgaggccgcccgggcaaagcccgggcgtcgggcgacctttggtc
gcccggcctcagtgagcgagcgagcgcgcagagagggagtggccaactccatcactaggggttcctgcggccgcacgcgccagc
tctgggtatttaagcccgagtgagcacgcagggtctccattttgaagcgggaggttacgcgttcgtcgactactagtgggtaccagagc
tccctaggttctagaaccggtgacgtctcccatggtgaagcttggatctgaattcggtacctagttattaatagtaatcaattacggggtca
ttagttcatagcccatatatggagttccgcgttacataacttacggtaaatggcccgcctggctgaccgcccaacgacccccgcccattg
acgtcaataatgacgtatgttcccatagtaacgccaatagggactttccattgacgtcaatgggtggagtatttacggtaaactgcccact
tggcagtacatcaagtgtatcatatgccaagtacgccccctattgacgtcaatgacggtaaatggcccgcctggcattatgcccagtaca
tgaccttatgggactttcctacttggcagtacatctacgtattagtcatcgctattaccatgggggtgtctgtattactgggcgaggtgtcct
cccttcctggggactgtggggggtggtcaaaagacctctatgccccacctccttcctccctctgccctgctgtgcctggggcaggggg
agaacagcccacctcgtgactgggggctggcccagcccgccctatccctgggggaggggggggacagggggagccctataatt
ggacaagtctgggatccttgagtcctggagtcgctgcgcgctgccttcgccccgtgccccgctccgccgccgcctcgcgccgcccg
ccccggctctgactgaccgcgttactcccacaggtgagcggggggacggcccttctcctccgggctgtaattagcgcttggtttaatg
acggcttgtttcttttctgtggctgcgtgaaagccttgaggggctccgggagctagagcctctgctaaccatgttcatgccttcttctttttcc
tacagctcctgggcaacgtgctggttattgtgctgtctcatcattttggcaaagaattcctcgaagatccgaagggaaagtcttccacgac
tgtgggatccgttcgaagatatcaccggttgagccaccatggtgagcaagggcgaggagctgttcaccggggtggtgcccatcctgg
tcgagctggacggcgacgtaaacggccacaagttcagcgtgtccggcgagggcgagggcgatgccacctacggcaagctgaccct
gaagttcatctgcaccaccggcaagctgcccgtgccctggcccaccctcgtgaccaccctgacctacggcgtgcagtgcttcagccg
ctaccccgaccacatgaagcagcacgacttcttcaagtccgccatgcccgaaggctacgtccaggagcgcaccatcttcttcaaggac
gacggcaactacaagacccgcgccgaggtgaagttcgagggcgacaccctggtgaaccgcatcgagctgaagggcatcgacttca
aggaggacggcaacatcctggggcacaagctggagtacaactacaacagccacaacgtctatatcatggccgacaagcagaagaa
cggcatcaaggtgaacttcaagatccgccacaacatcgaggacggcagcgtgcagctcgccgaccactaccagcagaacaccccc
atcggcgacggccccgtgctgctgcccgacaaccactacctgagcacccagtccgccctgagcaaagaccccaacgagaagcgc
gatcacatggtcctgctggagttcgtgaccgccgccgggatcactctcggcatggacgagctgtacaagtaacaattgttaattaagttt
aaaccctcgaggccgcaagcttatcgataatcaacctctggattacaaaatttgtgaaagattgactggtattcttaactatgttgctcctttt
acgctatgtggatacgctgctttaatgcctttgtatcatgctattgcttcccgtatggctttcattttctcctccttgtataaatcctggttgctgt
ctctttatgaggagttgtggcccgttgtcaggcaacgtggcgtggtgtgcactgtgtttgctgacgcaacccccactggttggggcattg
ccaccacctgtcagctcctttccgggactttcgctttccccctccctattgccacggcggaactcatcgccgcctgccttgcccgctgctg
gacaggggctcggctgttgggcactgacaattccgtggtgttgtcggggaaatcatcgtcctttccttggctgctcgcctgtgttgccac
ctggattctgcgcgggacgtccttctgctacgtcccttcggccctcaatccagcggaccttccttcccgcggcctgctgccggctctgc
ggcctcttccgcgtcttcgccttcgccctcagacgagtcggatctccctttgggccgcctccccgcatcgataccgtcgactagagctc
gctgatcagcctcgactgtgccttctagttgccagccatctgttgtttgcccctcccccgtgccttccttgaccctggaaggtgccactcc
cactgtcctttcctaataaaatgaggaaattgcatcgcattgtctgagtaggtgtcattctattctggggggtggggggggcaggacag
caagggggaggattgggaagacaatagcaggcatgctggggagagatccacgataacaaacagcttttttggggtgaacatattgac
tgaattcccgtgcggaccgagcggccgcaggaacccctagtgatggagttggccactccctctctgcgcgctcgctcgctcactgag
gccgggcgaccaaaggtcgcccgacgcccgggctttgcccgggggcctcagtgagcgagcgagcgcgcagctgcctgcaggc
atgcaagctgtagccaaccactagaactatagctagagtcctgggcgaacaaacgatgctcgccttccagaaaaccgaggatgcgaa
ccacttcatccggggtcagcaccaccggcaagcgccgcgacggccgaggtcttccgatctcctgaagccagggcagatccgtgcac
agcaccttgccgtagaagaacagcaaggccgccaatgcctgacgatgcgtggagaccgaaaccttgcgctcgttcgccagccagga
cagaaatgcctcgacttcgctgctgcccaaggttgccgggtgacgcacaccgtggaaacggatgaaggcacgaacccagttgacat
aagcctgttcggttcgtaaactgtaatgcaagtagcgtatgcgctcacgcaactggtccagaaccttgaccgaacgcagcggtggtaa
cggcgcagtggcggttttcatggcttgttatgactgtttttttgtacagtctatgcctcgggcatccaagcagcaagcgcgttacgccgtg
ggtcgatgtttgatgttatggagcagcaacgatgttacgcagcagcaacgatgttacgcagcagggcagtcgccctaaaacaaagtta
ggtggctcaagtatgggcatcattcgcacatgtaggctcggccctgaccaagtcaaatccatgcgggctgctcttgatcttttcggtcgt
gagttcggagacgtagccacctactcccaacatcagccggactccgattacctcgggaacttgctccgtagtaagacattcatcgcgct
tgctgccttcgaccaagaagcggttgttggcgctctcgcggcttacgttctgcccaggtttgagcagccgcgtagtgagatctatatctat
gatctcgcagtctccggcgagcaccggaggcagggcattgccaccgcgctcatcaatctcctcaagcatgaggccaacgcgcttggt
gcttatgtgatctacgtgcaagcagattacggtgacgatcccgcagtggctctctatacaaagttgggcatacgggaagaagtgatgca
ctttgatatcgacccaagtaccgccacctaacaattcgttcaagccgagatcggcttcccggccgcggagttgttcggtaaattgtcaca
acgccgcgaatatagtctttaccatgcccttggccacgcccctctttaatacgacgggcaatttgcacttcagaaaatgaagagtttgcttt
agccataacaaaagtccagtatgctttttcacagcataactggactgatttcagtttacaactattctgtctagtttaagactttattgtcatagt
ttagatctattttgttcagtttaagactttattgtccgcccacacccgcttacgcagggcatccatttattactcaaccgtaaccgattttgcca
ggttacgcggctggtctgcggtgtgaaataccgcacagatgcgtaaggagaaaataccgcatcaggcgctcttccgcttcctcgctca
ctgactcgctgcgctcggtcgttcggctgcggcgagcggtatcagctcactcaaaggcggtaatacggttatccacagaatcagggg
ataacgcaggaaagaacatgtgagcaaaaggccagcaaaaggccaggaaccgtaaaaaggccgcgttgctggcgtttttccatagg
ctccgcccccctgacgagcatcacaaaaatcgacgctcaagtcagaggtggcgaaacccgacaggactataaagataccaggcgttt
ccccctggaagctccctcgtgcgctctcctgttccgaccctgccgcttaccggatacctgtccgcctttctcccttcgggaagcgtggcg
ctttctcaatgctcacgctgtaggtatctcagttcggtgtaggtcgttcgctccaagctgggctgtgtgcacgaaccccccgttcagcccg
accgctgcgccttatccggtaactatcgtcttgagtccaacccggtaagacacgacttatcgccactggcagcagccactggtaacag
gattagcagagcgaggtatgtaggcggtgctacagagttcttgaagtggtggcctaactacggctacactagaaggacagtatttggta
tctgcgctctgctgaagccagttaccttcggaaaaagagttggtagctcttgatccggcaaacaaaccaccgctggtagcggtggtttttt
tgtttgcaagcagcagattacgcgcagaaaaaaaggatctcaagaagatcctttgatcttttctacggggtctgacgctcagtggaacga
aaactcacgttaagggattttggtcatgagattatcaaaaaggatcttcacctagatccttttaaattaaaaatgaagttttaaatcaatctaa
agtatatatgagtaaacttggtctgacagttaccaatgcttaatcagtgaggcacctatctcagcgatctgtctatttcgttcatccatagttg
cctgactccccgtcgtgtagataactacgatacgggagggcttaccatctggccccagtgctgcaatgataccgcgagacccacgctc
accggctccagatttatcagcaataaaccagccagccggaagggccgagcgcagaagtggtcctgcaactttatccgcctccatcca
gtctattaattgttgccgggaagctagagtaagtagttcgccagttaatagtttgcgcaacgttgttgccattgctacaggcatcgtggtgt
cacgctcgtcgtttggtatggcttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatggcagcactgcataattctcttactgt
catgccatccgtaagatgcttttctgtgactggtgagtactcaaccaagtcattctgagaatagtgtatgcggcgaccgagttgctcttgc
ccggcgtcaatacgggataataccgcgccacatagcagaactttaaaagtgctcatcattggaaaacgttcttcggggcgaaaactctc
aaggatcttaccgctgttgagatccagttcgatgtaacccactcgtgcacccaactgatcttcagcatcttttactttcaccagcgtttctgg
gtgagcaaaaacaggaaggcaaaatgccgcaaaaaagggaataagggcgacacggaaatgttgaatactcatactcttcctttttcaa
tattattgaagcatttatcagggttattgtctcatgagcggatacatatttgaatgtatttagaaaaataaacaaataggggttccgcgcaca
tttccccgaaaagtgccacctgaaattgtaaacgttaatattttgttaaaattcgcgttaaatttttgttaaatcagctcattttttaaccaatag
gccgaaatcggcaaaatcccttataaatcaaaagaatagaccgagatagggttgagtgttgttccagtttggaacaagagtccactatta
aagaacgtggactccaacgtcaaagggcgaaaaaccgtctatcagggcgatggcccactacgtgaaccatcaccctaatcaagtttttt
ggggtcgaggtgccgtaaagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgctgcgcgtaaccaccac
acccgccgcgcttaatgcgccgctacagggcgcgtc
CBA-GFP expression construct/vector (7313 nt)
SEQ ID NO: 14
cattcgccattcaggctgcaaataagcgttgatattcagtcaattacaaacattaataacgaagagatgacagaaaaattttcattctgtga
cagagaaaaagtagccgaagatgacggtttgtcacatggagttggcaggatgtttgattaaaaacataacaggaagaaaaatgccccg
ctgtgggcggacaaaatagttgggaactgggaggggtggaaatggagtttttaaggattatttagggaagagtgacaaaatagatggg
aactgggtgtagcgtcgtaagctaatacgaaaattaaaaatgacaaaatagtttggaactagatttcacttatctggttcggatctcctaga
gcttacagcttcctgcaggcagctgcgcgctcgctcgctcactgaggccgcccgggcaaagcccgggcgtcgggcgacctttggtc
gcccggcctcagtgagcgagcgagcgcgcagagagggagtggccaactccatcactaggggttcctgcggccgcacgcgttcgtc
gactactagtgggtaccagagctccctaggttctagaaccggtgacgtctcccatggtgaagcttggatctgaattcggtacctagttatt
aatagtaatcaattacggggtcattagttcatagcccatatatggagttccgcgttacataacttacggtaaatggcccgcctggctgacc
gcccaacgacccccgcccattgacgtcaataatgacgtatgttcccatagtaacgccaatagggactttccattgacgtcaatgggtgg
agtatttacggtaaactgcccacttggcagtacatcaagtgtatcatatgccaagtacgccccctattgacgtcaatgacggtaaatggc
ccgcctggcattatgcccagtacatgaccttatgggactttcctacttggcagtacatctacgtattagtcatcgctattaccatggtcgag
gtgagccccacgttctgcttcactctccccatctcccccccctccccacccccaattttgtatttatttattttttaattattttgtgcagcgatg
ggggcggggggggggggggggcgcgcgccaggcggggcggggcggggcgaggggggggcggggcgaggcggagagg
tgcggcggcagccaatcagagcggcgcgctccgaaagtttccttttatggcgaggcggcggcggcggcggccctataaaaagcga
agcgcgcggggggggagtcgctgcgcgctgccttcgccccgtgccccgctccgccgccgcctcgcgccgcccgccccggctc
tgactgaccgcgttactcccacaggtgagcggggggacggcccttctcctccgggctgtaattagcgcttggtttaatgacggcttgtt
tcttttctgtggctgcgtgaaagccttgaggggctccgggagctagagcctctgctaaccatgttcatgccttcttctttttcctacagctcc
tgggcaacgtgctggttattgtgctgtctcatcattttggcaaagaattcctcgaagatccgaagggaaagtcttccacgactgtgggatc
cgccaccatggtgagcaagggcgaggagctgttcaccggggtggtgcccatcctggtcgagctggacggcgacgtaaacggccac
aagttcagcgtgtccggcgagggcgagggcgatgccacctacggcaagctgaccctgaagttcatctgcaccaccggcaagctgcc
cgtgccctggcccaccctcgtgaccaccctgacctacggcgtgcagtgcttcagccgctaccccgaccacatgaagcagcacgactt
cttcaagtccgccatgcccgaaggctacgtccaggagcgcaccatcttcttcaaggacgacggcaactacaagacccgcgccgagg
tgaagttcgagggcgacaccctggtgaaccgcatcgagctgaagggcatcgacttcaaggaggacggcaacatcctggggcacaa
gctggagtacaactacaacagccacaacgtctatatcatggccgacaagcagaagaacggcatcaaggtgaacttcaagatccgcca
caacatcgaggacggcagcgtgcagctcgccgaccactaccagcagaacacccccatcggcgacggccccgtgctgctgcccga
caaccactacctgagcacccagtccgccctgagcaaagaccccaacgagaagcgcgatcacatggtcctgctggagttcgtgaccg
ccgccgggatcactctcggcatggacgagctgtacaagtaacaattgttaattaagtttaaaccctcgaggccgcaagcttatcgataat
caacctctggattacaaaatttgtgaaagattgactggtattcttaactatgttgctccttttacgctatgtggatacgctgctttaatgcctttg
tatcatgctattgcttcccgtatggctttcattttctcctccttgtataaatcctggttgctgtctctttatgaggagttgtggcccgttgtcaggc
aacgtggcgtggtgtgcactgtgtttgctgacgcaacccccactggttggggcattgccaccacctgtcagctcctttccgggactttcg
ctttccccctccctattgccacggcggaactcatcgccgcctgccttgcccgctgctggacaggggctcggctgttgggcactgacaat
tccgtggtgttgtcggggaaatcatcgtcctttccttggctgctcgcctgtgttgccacctggattctgcgcgggacgtccttctgctacgt
cccttcggccctcaatccagcggaccttccttcccgcggcctgctgccggctctgcggcctcttccgcgtcttcgccttcgccctcagac
gagtcggatctccctttgggccgcctccccgcatcgataccgtcgactagagctcgctgatcagcctcgactgtgccttctagttgccag
ccatctgttgtttgcccctcccccgtgccttccttgaccctggaaggtgccactcccactgtcctttcctaataaaatgaggaaattgcatc
gcattgtctgagtaggtgtcattctattctggggggtggggggggcaggacagcaagggggaggattgggaagacaatagcaggc
atgctggggagagatccacgataacaaacagcttttttggggtgaacatattgactgaattcccgtgcggaccgagcggccgcaggaa
cccctagtgatggagttggccactccctctctgcgcgctcgctcgctcactgaggccgggcgaccaaaggtcgcccgacgcccggg
ctttgcccgggcggcctcagtgagcgagcgagcgcgcagctgcctgcaggcatgcaagctgtagccaaccactagaactatagcta
gagtcctgggcgaacaaacgatgctcgccttccagaaaaccgaggatgcgaaccacttcatccggggtcagcaccaccggcaagc
gccgcgacggccgaggtcttccgatctcctgaagccagggcagatccgtgcacagcaccttgccgtagaagaacagcaaggccgc
caatgcctgacgatgcgtggagaccgaaaccttgcgctcgttcgccagccaggacagaaatgcctcgacttcgctgctgcccaaggtt
gccgggtgacgcacaccgtggaaacggatgaaggcacgaacccagttgacataagcctgttcggttcgtaaactgtaatgcaagtag
cgtatgcgctcacgcaactggtccagaaccttgaccgaacgcagcggtggtaacggcgcagtggcggttttcatggcttgttatgactg
tttttttgtacagtctatgcctcgggcatccaagcagcaagcgcgttacgccgtgggtcgatgtttgatgttatggagcagcaacgatgtt
acgcagcagcaacgatgttacgcagcagggcagtcgccctaaaacaaagttaggtggctcaagtatgggcatcattcgcacatgtag
gctcggccctgaccaagtcaaatccatgcgggctgctcttgatcttttcggtcgtgagttcggagacgtagccacctactcccaacatca
gccggactccgattacctcgggaacttgctccgtagtaagacattcatcgcgcttgctgccttcgaccaagaagcggttgttggcgctct
cgcggcttacgttctgcccaggtttgagcagccgcgtagtgagatctatatctatgatctcgcagtctccggcgagcaccggaggcag
ggcattgccaccgcgctcatcaatctcctcaagcatgaggccaacgcgcttggtgcttatgtgatctacgtgcaagcagattacggtga
cgatcccgcagtggctctctatacaaagttgggcatacgggaagaagtgatgcactttgatatcgacccaagtaccgccacctaacaat
tcgttcaagccgagatcggcttcccggccgcggagttgttcggtaaattgtcacaacgccgcgaatatagtctttaccatgcccttggcc
acgcccctctttaatacgacgggcaatttgcacttcagaaaatgaagagtttgctttagccataacaaaagtccagtatgctttttcacagc
ataactggactgatttcagtttacaactattctgtctagtttaagactttattgtcatagtttagatctattttgttcagtttaagactttattgtccg
cccacacccgcttacgcagggcatccatttattactcaaccgtaaccgattttgccaggttacgcggctggtctgcggtgtgaaataccg
cacagatgcgtaaggagaaaataccgcatcaggcgctcttccgcttcctcgctcactgactcgctgcgctcggtcgttcggctgcggc
gagcggtatcagctcactcaaaggcggtaatacggttatccacagaatcaggggataacgcaggaaagaacatgtgagcaaaaggc
cagcaaaaggccaggaaccgtaaaaaggccgcgttgctggcgtttttccataggctccgcccccctgacgagcatcacaaaaatcga
cgctcaagtcagaggtggcgaaacccgacaggactataaagataccaggcgtttccccctggaagctccctcgtgcgctctcctgttc
cgaccctgccgcttaccggatacctgtccgcctttctcccttcgggaagcgtggcgctttctcaatgctcacgctgtaggtatctcagttc
ggtgtaggtcgttcgctccaagctgggctgtgtgcacgaaccccccgttcagcccgaccgctgcgccttatccggtaactatcgtcttg
agtccaacccggtaagacacgacttatcgccactggcagcagccactggtaacaggattagcagagcgaggtatgtaggcggtgcta
cagagttcttgaagtggtggcctaactacggctacactagaaggacagtatttggtatctgcgctctgctgaagccagttaccttcggaa
aaagagttggtagctcttgatccggcaaacaaaccaccgctggtagcggtggtttttttgtttgcaagcagcagattacgcgcagaaaa
aaaggatctcaagaagatcctttgatcttttctacggggtctgacgctcagtggaacgaaaactcacgttaagggattttggtcatgagat
tatcaaaaaggatcttcacctagatccttttaaattaaaaatgaagttttaaatcaatctaaagtatatatgagtaaacttggtctgacagttac
caatgcttaatcagtgaggcacctatctcagcgatctgtctatttcgttcatccatagttgcctgactccccgtcgtgtagataactacgata
cgggagggcttaccatctggccccagtgctgcaatgataccgcgagacccacgctcaccggctccagatttatcagcaataaaccag
ccagccggaagggccgagcgcagaagtggtcctgcaactttatccgcctccatccagtctattaattgttgccgggaagctagagtaa
gtagttcgccagttaatagtttgcgcaacgttgttgccattgctacaggcatcgtggtgtcacgctcgtcgtttggtatggcttcattcagct
ccggttcccaacgatcaaggcgagttacatgatcccccatgttgtgcaaaaaagcggttagctccttcggtcctccgatcgttgtcagaa
gtaagttggccgcagtgttatcactcatggttatggcagcactgcataattctcttactgtcatgccatccgtaagatgcttttctgtgactg
gtgagtactcaaccaagtcattctgagaatagtgtatgcggcgaccgagttgctcttgcccggcgtcaatacgggataataccgcgcca
catagcagaactttaaaagtgctcatcattggaaaacgttcttcggggcgaaaactctcaaggatcttaccgctgttgagatccagttcga
tgtaacccactcgtgcacccaactgatcttcagcatcttttactttcaccagcgtttctgggtgagcaaaaacaggaaggcaaaatgccg
caaaaaagggaataagggcgacacggaaatgttgaatactcatactcttcctttttcaatattattgaagcatttatcagggttattgtctcat
gagcggatacatatttgaatgtatttagaaaaataaacaaataggggttccgcgcacatttccccgaaaagtgccacctgaaattgtaaa
cgttaatattttgttaaaattcgcgttaaatttttgttaaatcagctcattttttaaccaataggccgaaatcggcaaaatcccttataaatcaaa
agaatagaccgagatagggttgagtgttgttccagtttggaacaagagtccactattaaagaacgtggactccaacgtcaaagggcga
aaaaccgtctatcagggcgatggcccactacgtgaaccatcaccctaatcaagttttttggggtcgaggtgccgtaaagcactaaatcg
gaaccctaaagggagcccccgatttagagcttgacggggaaagccggcgaacgtggcgagaaaggaagggaagaaagcgaaag
gagcgggcgctagggcgctggcaagtgtagcggtcacgctgcgcgtaaccaccacacccgccgcgcttaatgcgccgctacaggg
cgcgtc
ApoP-GFP expression construct/vector (7047 nt)
SEQ ID NO: 15
cattcgccattcaggctgcaaataagcgttgatattcagtcaattacaaacattaataacgaagagatgacagaaaaattttcattctgtga
cagagaaaaagtagccgaagatgacggtttgtcacatggagttggcaggatgtttgattaaaaacataacaggaagaaaaatgccccg
ctgtgggcggacaaaatagttgggaactgggaggggtggaaatggagtttttaaggattatttagggaagagtgacaaaatagatggg
aactgggtgtagcgtcgtaagctaatacgaaaattaaaaatgacaaaatagtttggaactagatttcacttatctggttcggatctcctaga
gcttacagcttcctgcaggcagctgcgcgctcgctcgctcactgaggccgcccgggcaaagcccgggcgtcgggcgacctttggtc
gcccggcctcagtgagcgagcgagcgcgcagagagggagtggccaactccatcactaggggttcctgcggccgcacgcgccagc
tctgggtatttaagcccgagtgagcacgcagggtctccattttgaagcgggaggttacgcgttcgtcgactactagtgggtaccagagc
tccctaggttctagaaccggtgacgtctcccatggtgaagcttggatctgaattcggtacctagttattaatagtaatcaattacggggtca
ttagttcatagcccatatatggagttccgggggtgtctgtattactgggcgaggtgtcctcccttcctggggactgtggggggtggtcaa
aagacctctatgccccacctccttcctccctctgccctgctgtgcctggggcagggggagaacagcccacctcgtgactgggggctg
gcccagcccgccctatccctgggggaggggggggacagggggagccctataattggacaagtctgggatccttgagtcctggagt
cgctgcgcgctgccttcgccccgtgccccgctccgccgccgcctcgcgccgcccgccccggctctgactgaccgcgttactcccac
aggtgagcgggcgggacggcccttctcctccgggctgtaattagcgcttggtttaatgacggcttgtttcttttctgtggctgcgtgaaag
ccttgaggggctccgggagctagagcctctgctaaccatgttcatgccttcttctttttcctacagctcctgggcaacgtgctggttattgt
gctgtctcatcattttggcaaagaattcctcgaagatccgaagggaaagtcttccacgactgtgggatccgttcgaagatatcaccggtt
gagccaccatggtgagcaagggcgaggagctgttcaccggggtggtgcccatcctggtcgagctggacggcgacgtaaacggcca
caagttcagcgtgtccggcgagggcgagggcgatgccacctacggcaagctgaccctgaagttcatctgcaccaccggcaagctgc
ccgtgccctggcccaccctcgtgaccaccctgacctacggcgtgcagtgcttcagccgctaccccgaccacatgaagcagcacgact
tcttcaagtccgccatgcccgaaggctacgtccaggagcgcaccatcttcttcaaggacgacggcaactacaagacccgcgccgag
gtgaagttcgagggcgacaccctggtgaaccgcatcgagctgaagggcatcgacttcaaggaggacggcaacatcctggggcaca
agctggagtacaactacaacagccacaacgtctatatcatggccgacaagcagaagaacggcatcaaggtgaacttcaagatccgcc
acaacatcgaggacggcagcgtgcagctcgccgaccactaccagcagaacacccccatcggcgacggccccgtgctgctgcccg
acaaccactacctgagcacccagtccgccctgagcaaagaccccaacgagaagcgcgatcacatggtcctgctggagttcgtgacc
gccgccgggatcactctcggcatggacgagctgtacaagtaacaattgttaattaagtttaaaccctcgaggccgcaagcttatcgata
atcaacctctggattacaaaatttgtgaaagattgactggtattcttaactatgttgctccttttacgctatgtggatacgctgctttaatgcctt
tgtatcatgctattgcttcccgtatggctttcattttctcctccttgtataaatcctggttgctgtctctttatgaggagttgtggcccgttgtcag
gcaacgtggcgtggtgtgcactgtgtttgctgacgcaacccccactggttggggcattgccaccacctgtcagctcctttccgggacttt
cgctttccccctccctattgccacggcggaactcatcgccgcctgccttgcccgctgctggacaggggctcggctgttgggcactgac
aattccgtggtgttgtcggggaaatcatcgtcctttccttggctgctcgcctgtgttgccacctggattctgcgcgggacgtccttctgcta
cgtcccttcggccctcaatccagcggaccttccttcccgcggcctgctgccggctctgcggcctcttccgcgtcttcgccttcgccctca
gacgagtcggatctccctttgggccgcctccccgcatcgataccgtcgactagagctcgctgatcagcctcgactgtgccttctagttgc
cagccatctgttgtttgcccctcccccgtgccttccttgaccctggaaggtgccactcccactgtcctttcctaataaaatgaggaaattgc
atcgcattgtctgagtaggtgtcattctattctggggggtggggggggcaggacagcaagggggaggattgggaagacaatagcag
gcatgctggggagagatccacgataacaaacagcttttttggggtgaacatattgactgaattcccgtgcggaccgagcggccgcag
gaacccctagtgatggagttggccactccctctctgcgcgctcgctcgctcactgaggccgggcgaccaaaggtcgcccgacgccc
gggctttgcccgggcggcctcagtgagcgagcgagcgcgcagctgcctgcaggcatgcaagctgtagccaaccactagaactata
gctagagtcctgggcgaacaaacgatgctcgccttccagaaaaccgaggatgcgaaccacttcatccggggtcagcaccaccggca
agcgccgcgacggccgaggtcttccgatctcctgaagccagggcagatccgtgcacagcaccttgccgtagaagaacagcaaggc
cgccaatgcctgacgatgcgtggagaccgaaaccttgcgctcgttcgccagccaggacagaaatgcctcgacttcgctgctgcccaa
ggttgccgggtgacgcacaccgtggaaacggatgaaggcacgaacccagttgacataagcctgttcggttcgtaaactgtaatgcaa
gtagcgtatgcgctcacgcaactggtccagaaccttgaccgaacgcagcggtggtaacggcgcagtggcggttttcatggcttgttat
gactgtttttttgtacagtctatgcctcgggcatccaagcagcaagcgcgttacgccgtgggtcgatgtttgatgttatggagcagcaacg
atgttacgcagcagcaacgatgttacgcagcagggcagtcgccctaaaacaaagttaggtggctcaagtatgggcatcattcgcacat
gtaggctcggccctgaccaagtcaaatccatgcgggctgctcttgatcttttcggtcgtgagttcggagacgtagccacctactcccaac
atcagccggactccgattacctcgggaacttgctccgtagtaagacattcatcgcgcttgctgccttcgaccaagaagcggttgttggc
gctctcgcggcttacgttctgcccaggtttgagcagccgcgtagtgagatctatatctatgatctcgcagtctccggcgagcaccggag
gcagggcattgccaccgcgctcatcaatctcctcaagcatgaggccaacgcgcttggtgcttatgtgatctacgtgcaagcagattacg
gtgacgatcccgcagtggctctctatacaaagttgggcatacgggaagaagtgatgcactttgatatcgacccaagtaccgccacctaa
caattcgttcaagccgagatcggcttcccggccgcggagttgttcggtaaattgtcacaacgccgcgaatatagtctttaccatgccctt
ggccacgcccctctttaatacgacgggcaatttgcacttcagaaaatgaagagtttgctttagccataacaaaagtccagtatgctttttca
cagcataactggactgatttcagtttacaactattctgtctagtttaagactttattgtcatagtttagatctattttgttcagtttaagactttattg
tccgcccacacccgcttacgcagggcatccatttattactcaaccgtaaccgattttgccaggttacgcggctggtctgcggtgtgaaat
accgcacagatgcgtaaggagaaaataccgcatcaggcgctcttccgcttcctcgctcactgactcgctgcgctcggtcgttcggctg
cggcgagcggtatcagctcactcaaaggcggtaatacggttatccacagaatcaggggataacgcaggaaagaacatgtgagcaaa
aggccagcaaaaggccaggaaccgtaaaaaggccgcgttgctggcgtttttccataggctccgcccccctgacgagcatcacaaaa
atcgacgctcaagtcagaggtggcgaaacccgacaggactataaagataccaggcgtttccccctggaagctccctcgtgcgctctc
ctgttccgaccctgccgcttaccggatacctgtccgcctttctcccttcgggaagcgtggcgctttctcaatgctcacgctgtaggtatctc
agttcggtgtaggtcgttcgctccaagctgggctgtgtgcacgaaccccccgttcagcccgaccgctgcgccttatccggtaactatcg
tcttgagtccaacccggtaagacacgacttatcgccactggcagcagccactggtaacaggattagcagagcgaggtatgtaggcgg
tgctacagagttcttgaagtggtggcctaactacggctacactagaaggacagtatttggtatctgcgctctgctgaagccagttaccttc
ggaaaaagagttggtagctcttgatccggcaaacaaaccaccgctggtagcggtggtttttttgtttgcaagcagcagattacgcgcag
aaaaaaaggatctcaagaagatcctttgatcttttctacggggtctgacgctcagtggaacgaaaactcacgttaagggattttggtcatg
agattatcaaaaaggatcttcacctagatccttttaaattaaaaatgaagttttaaatcaatctaaagtatatatgagtaaacttggtctgaca
gttaccaatgcttaatcagtgaggcacctatctcagcgatctgtctatttcgttcatccatagttgcctgactccccgtcgtgtagataacta
cgatacgggagggcttaccatctggccccagtgctgcaatgataccgcgagacccacgctcaccggctccagatttatcagcaataaa
ccagccagccggaagggccgagcgcagaagtggtcctgcaactttatccgcctccatccagtctattaattgttgccgggaagctaga
gtaagtagttcgccagttaatagtttgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctccttcggtcctccgatcgttgtc
agaagtaagttggccgcagtgttatcactcatggttatggcagcactgcataattctcttactgtcatgccatccgtaagatgcttttctgtg
actggtgagtactcaaccaagtcattctgagaatagtgtatgcggcgaccgagttgctcttgcccggcgtcaatacgggataataccgc
gccacatagcagaactttaaaagtgctcatcattggaaaacgttcttcggggcgaaaactctcaaggatcttaccgctgttgagatccag
ttcgatgtaacccactcgtgcacccaactgatcttcagcatcttttactttcaccagcgtttctgggtgagcaaaaacaggaaggcaaaat
gccgcaaaaaagggaataagggcgacacggaaatgttgaatactcatactcttcctttttcaatattattgaagcatttatcagggttattgt
ctcatgagcggatacatatttgaatgtatttagaaaaataaacaaataggggttccgcgcacatttccccgaaaagtgccacctgaaattg
taaacgttaatattttgttaaaattcgcgttaaatttttgttaaatcagctcattttttaaccaataggccgaaatcggcaaaatcccttataaat
caaaagaatagaccgagatagggttgagtgttgttccagtttggaacaagagtccactattaaagaacgtggactccaacgtcaaagg
gcgaaaaaccgtctatcagggcgatggcccactacgtgaaccatcaccctaatcaagttttttggggtcgaggtgccgtaaagcactaa
atcggaaccctaaagggagcccccgatttagagcttgacggggaaagccggcgaacgtggcgagaaaggaagggaagaaagcg
aaaggagcgggcgctagggcgctggcaagtgtagcggtcacgctgcgcgtaaccaccacacccgccgcgcttaatgcgccgctac
agggcgcgtc
U6 promoter (264 nt)
SEQ ID NO: 16
aaggtcgggcaggaagagggcctatttcccatgattccttcatatttgcatatacgatacaaggctgttagagagataattagaattaattt
gactgtaaacacaaagatattagtacaaaatacgtgacgtagaaagtaataatttcttgggtagtttgcagttttaaaattatgttttaaaatg
gactatcatatgcttaccgtaacttgaaagtatttcgatttcttggctttatatatcttgtggaaaggacgaaacacc
ES01(-1) guide strand (21 nt)
SEQ ID NO: 17
ctgcacccagcgcaggtaatc
ES07 guide strand (21 nt)
SEQ ID NO: 18
tttattaaactagggtccacc
APOE_shES01(-1) (54 nt)
SEQ ID NO: 19
caccgattacctgcgctgggtgcagcgaactgcacccagcgcaggtaatctttt
eSibr_ApoE_ES07c_sh (150 nt)
SEQ ID NO: 20
ctggaggcttgctttgggctgtatgctgtttattaaactagggtccaccttttggcctctgactgaggtggaccctagtttaataaacagga
cacaaggccctttatcagcactcacatggaacaaatggccaccgtgggaggatgacaa
U6_APOE_shES01(-1)_CAPO_optAPOE2 (7451 nt)
SEQ ID NO: 21
cattcgccattcaggctgcaaataagcgttgatattcagtcaattacaaacattaataacgaagagatgacagaaaaattttcattctgtga
cagagaaaaagtagccgaagatgacggtttgtcacatggagttggcaggatgtttgattaaaaacataacaggaagaaaaatgccccg
ctgtgggcggacaaaatagttgggaactgggaggggtggaaatggagtttttaaggattatttagggaagagtgacaaaatagatggg
aactgggtgtagcgtcgtaagctaatacgaaaattaaaaatgacaaaatagtttggaactagatttcacttatctggttcggatctcctaga
gcttacagcttcctgcaggcagctgcgcgctcgctcgctcactgaggccgcccgggcaaagcccgggcgtcgggcgacctttggtc
gcccggcctcagtgagcgagcgagcgcgcagagagggagtggccaactccatcactaggggttcctgcggccgcacgcgtgttac
taaggtcgggcaggaagagggcctatttcccatgattccttcatatttgcatatacgatacaaggctgttagagagataattagaattaatt
tgactgtaaacacaaagatattagtacaaaatacgtgacgtagaaagtaataatttcttgggtagtttgcagttttaaaattatgttttaaaat
ggactatcatatgcttaccgtaacttgaaagtatttcgatttcttggctttatatatcttgtggaaaggacgaaacacccaccgattacctgc
gctgggtgcagcgaactgcacccagcgcaggtaatcttttttttttaggttctagaaccggtgacgtctcccatggtgaagcttggatctg
aattcggtacctagttattaatagtaatcaattacggggtcattagttcatagcccatatatggagttccgcgttacataacttacggtaaatg
gcccgcctggctgaccgcccaacgacccccgcccattgacgtcaataatgacgtatgttcccatagtaacgccaatagggactttccat
tgacgtcaatgggtggagtatttacggtaaactgcccacttggcagtacatcaagtgtatcatatgccaagtacgccccctattgacgtca
atgacggtaaatggcccgcctggcattatgcccagtacatgaccttatgggactttcctacttggcagtacatctacgtattagtcatcgct
attaccatgggggtgtctgtattactgggcgaggtgtcctcccttcctggggactgtggggggggtcaaaagacctctatgccccacct
ccttcctccctctgccctgctgtgcctggggcagggggagaacagcccacctcgtgactgggggctggcccagcccgccctatccct
gggggagggggcgggacagggggagccctataattggacaagtctgggatccttgagtcctgccaccatgaaggttctgtgggctg
ccttgcttgtgacattcctggctggatgccaggccaaagtggagcaggctgtggaaacagaacctgagcctgaactgaggcagcaga
cagagtggcagtctggccagagatgggaactggctttgggaaggttctgggactacctgagatgggtgcagaccctgtctgaacagg
tgcaggaggagctgctgtctagccaagtgacccaagaactgagagccctgatggatgagaccatgaaggagctgaaggcctacaag
tctgagctggaggagcaactcacaccagtggctgaggagacaagggctaggctgagcaaggaattgcaagctgctcaggccaggtt
gggagcagacatggaggatgtgtgtggcagactggtgcagtacaggggagaggttcaggctatgcttggccagagcacagaggaa
ctgagggtgaggcttgctagccacctcaggaagctgaggaagaggctgctgagagatgctgatgacctccagaagtgcctggctgtg
tatcaagctggagctagggagggagctgaaaggggactgtctgccatcagggaaagactgggacctctggttgagcagggaagagt
gagagctgccacagtgggatctttggctggccaacctctgcaagaaagggctcaagcttggggagagaggcttagggccagaatgg
aggagatgggaagcaggacaagggacaggctggatgaggtgaaggagcaagtggctgaagtgagggccaagctggaagaacag
gcccagcagattagactgcaggctgaagccttccaagccaggctgaagagctggtttgagcccttggtggaggatatgcaaaggcag
tgggctggcctggttgagaaagtgcaagctgctgttggcacctctgctgctcctgtgcccagtgacaaccactgacaattgttaattaag
tttaaaccctcgaggccgcaagcttatcgataatcaacctctggattacaaaatttgtgaaagattgactggtattcttaactatgttgctcct
tttacgctatgtggatacgctgctttaatgcctttgtatcatgctattgcttcccgtatggctttcattttctcctccttgtataaatcctggttgct
gtctctttatgaggagttgtggcccgttgtcaggcaacgtggcgtggtgtgcactgtgtttgctgacgcaacccccactggttggggcat
tgccaccacctgtcagctcctttccgggactttcgctttccccctccctattgccacggcggaactcatcgccgcctgccttgcccgctgc
tggacaggggctcggctgttgggcactgacaattccgtggtgttgtcggggaaatcatcgtcctttccttggctgctcgcctgtgttgcca
cctggattctgcgcgggacgtccttctgctacgtcccttcggccctcaatccagcggaccttccttcccgcggcctgctgccggctctgc
ggcctcttccgcgtcttcgccttcgccctcagacgagtcggatctccctttgggccgcctccccgcatcgataccgtcgactagagctc
gctgatcagcctcgactgtgccttctagttgccagccatctgttgtttgcccctcccccgtgccttccttgaccctggaaggtgccactcc
cactgtcctttcctaataaaatgaggaaattgcatcgcattgtctgagtaggtgtcattctattctggggggtggggtggggcaggacag
caagggggaggattgggaagacaatagcaggcatgctggggagagatccacgataacaaacagcttttttggggtgaacatattgac
tgaattcccgtgcggaccgagcggccgcaggaacccctagtgatggagttggccactccctctctgcgcgctcgctcgctcactgag
gccgggcgaccaaaggtcgcccgacgcccgggctttgcccgggcggcctcagtgagcgagcgagcgcgcagctgcctgcaggc
atgcaagctgtagccaaccactagaactatagctagagtcctgggcgaacaaacgatgctcgccttccagaaaaccgaggatgcgaa
ccacttcatccggggtcagcaccaccggcaagcgccgcgacggccgaggtcttccgatctcctgaagccagggcagatccgtgcac
agcaccttgccgtagaagaacagcaaggccgccaatgcctgacgatgcgtggagaccgaaaccttgcgctcgttcgccagccagga
cagaaatgcctcgacttcgctgctgcccaaggttgccgggtgacgcacaccgtggaaacggatgaaggcacgaacccagttgacat
aagcctgttcggttcgtaaactgtaatgcaagtagcgtatgcgctcacgcaactggtccagaaccttgaccgaacgcagcggtggtaa
cggcgcagtggcggttttcatggcttgttatgactgtttttttgtacagtctatgcctcgggcatccaagcagcaagcgcgttacgccgtg
ggtcgatgtttgatgttatggagcagcaacgatgttacgcagcagcaacgatgttacgcagcagggcagtcgccctaaaacaaagtta
ggtggctcaagtatgggcatcattcgcacatgtaggctcggccctgaccaagtcaaatccatgcgggctgctcttgatcttttcggtcgt
gagttcggagacgtagccacctactcccaacatcagccggactccgattacctcgggaacttgctccgtagtaagacattcatcgcgct
tgctgccttcgaccaagaagcggttgttggcgctctcgcggcttacgttctgcccaggtttgagcagccgcgtagtgagatctatatctat
gatctcgcagtctccggcgagcaccggaggcagggcattgccaccgcgctcatcaatctcctcaagcatgaggccaacgcgcttggt
gcttatgtgatctacgtgcaagcagattacggtgacgatcccgcagtggctctctatacaaagttgggcatacgggaagaagtgatgca
ctttgatatcgacccaagtaccgccacctaacaattcgttcaagccgagatcggcttcccggccgcggagttgttcggtaaattgtcaca
acgccgcgaatatagtctttaccatgcccttggccacgcccctctttaatacgacgggcaatttgcacttcagaaaatgaagagtttgcttt
agccataacaaaagtccagtatgctttttcacagcataactggactgatttcagtttacaactattctgtctagtttaagactttattgtcatagt
ttagatctattttgttcagtttaagactttattgtccgcccacacccgcttacgcagggcatccatttattactcaaccgtaaccgattttgcca
ggttacgcggctggtctgcggtgtgaaataccgcacagatgcgtaaggagaaaataccgcatcaggcgctcttccgcttcctcgctca
ctgactcgctgcgctcggtcgttcggctgcggcgagcggtatcagctcactcaaaggcggtaatacggttatccacagaatcagggg
ataacgcaggaaagaacatgtgagcaaaaggccagcaaaaggccaggaaccgtaaaaaggccgcgttgctggcgtttttccatagg
ctccgcccccctgacgagcatcacaaaaatcgacgctcaagtcagaggtggcgaaacccgacaggactataaagataccaggcgttt
ccccctggaagctccctcgtgcgctctcctgttccgaccctgccgcttaccggatacctgtccgcctttctcccttcgggaagcgtggcg
ctttctcaatgctcacgctgtaggtatctcagttcggtgtaggtcgttcgctccaagctgggctgtgtgcacgaaccccccgttcagcccg
accgctgcgccttatccggtaactatcgtcttgagtccaacccggtaagacacgacttatcgccactggcagcagccactggtaacag
gattagcagagcgaggtatgtaggcggtgctacagagttcttgaagtggtggcctaactacggctacactagaaggacagtatttggta
tctgcgctctgctgaagccagttaccttcggaaaaagagttggtagctcttgatccggcaaacaaaccaccgctggtagcggtggtttttt
tgtttgcaagcagcagattacgcgcagaaaaaaaggatctcaagaagatcctttgatcttttctacggggtctgacgctcagtggaacga
aaactcacgttaagggattttggtcatgagattatcaaaaaggatcttcacctagatccttttaaattaaaa atgaagttttaaatcaatctaa
agtatatatgagtaaacttggtctgacagttaccaatgcttaatcagtgaggcacctatctcagcgatctgtctatttcgttcatccatagttg
cctgactccccgtcgtgtagataactacgatacgggagggcttaccatctggccccagtgctgcaatgataccgcgagacccacgctc
accggctccagatttatcagcaataaaccagccagccggaagggccgagcgcagaagtggtcctgcaactttatccgcctccatcca
gtctattaattgttgccgggaagctagagtaagtagttcgccagttaatagtttgcgcaacgttgttgccattgctacaggcatcgtggtgt
cacgctcgtcgtttggtatggcttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatggcagca ctgcataattctcttactgt
catgccatccgtaagatgcttttctgtgactggtgagtactcaaccaagtcattctgagaatagtgtatgcggcgaccgagttgctcttgc
ccggcgtcaatacgggataataccgcgccacatagcagaactttaaaagtgctcatcattggaaaacgttcttcggggcgaaaactctc
aaggatcttaccgctgttgagatccagttcgatgtaacccactcgtgcacccaactgatcttcagcatcttttactttcaccagcgtttctgg
gtgagcaaaaacaggaaggcaaaatgccgcaaaaaagggaataagggcgacacggaaatgttgaatactcatactcttcctttttcaa
tattattgaagcatttatcagggttattgtctcatgagcggatacatatttgaatgtatttagaaaaataaacaaataggggttccgcgcaca
tttccccgaaaagtgccacctgaaattgtaaacgttaatattttgttaaaattcgcgttaaatttttgttaaatcagctcattttttaaccaatag
gccgaaatcggcaaaatcccttataaatcaaaagaatagaccgagatagggttgagtgttgttccagtttggaacaagagtccactatta
aagaacgtggactccaacgtcaaagggcgaaaaaccgtctatcagggcgatggcccactacgtgaaccatcaccctaatcaagtttttt
ggggtcgaggtgccgtaaagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgctgcgcgtaaccaccac
acccgccgcgcttaatgcgccgctacagggcgcgtc
CAPO_eSibr_APOE_ES07c_sh_optAPOE2 (7640 nt)
SEQ ID NO: 22
cattcgccattcaggctgcaaataagcgttgatattcagtcaattacaaacattaataacgaagagatgacagaaaaattttcattctgtga
cagagaaaaagtagccgaagatgacggtttgtcacatggagttggcaggatgtttgattaaaaacataacaggaagaaaaatgccccg
ctgtgggcggacaaaatagttgggaactgggaggggtggaaatggagtttttaaggattatttagggaagagtgacaaaatagatggg
aactgggtgtagcgtcgtaagctaatacgaaaattaaaaatgacaaaatagtttggaactagatttcacttatctggttcggatctcctaga
gcttacagcttcctgcaggcagctgcgcgctcgctcgctcactgaggccgcccgggcaaagcccgggcgtcgggcgacctttggtc
gcccggcctcagtgagcgagcgagcgcgcagagagggagtggccaactccatcactaggggttcctgcggccgcacgcgtgttac
taggttctagaaccggtgacgtctcccatggtgaagcttggatctgaattcggtacctagttattaatagtaatcaattacggggtcattagt
tcatagcccatatatggagttccgcgttacataacttacggtaaatggcccgcctggctgaccgcccaacgacccccgcccattgacgt
caataatgacgtatgttcccatagtaacgccaatagggactttccattgacgtcaatgggtggagtatttacggtaaactgcccacttggc
agtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatgggggtgtctgtattactgggcgaggtgtcctccctt
cctggggactgtggggggtggtcaaaagacctctatgccccacctccttcctccctctgccctgctgtgcctggggcagggggagaa
cagcccacctcgtgactgggggctggcccagcccgccctatccctgggggaggggggggacagggggagccctataattggac
aagtctgggatccttgagtcctggagtcgctgcgacgctgccttcgccccgtgccccgctccgccgccgcctcgcgccgcccgcccc
ggctctgactgaccgcgttactcccacaggtgagcggggggacggcccttctcctccgggctgtaattagcgcttggtttaatgacg
gcttgtctggaggcttgctttgggctgtatgctgtttattaaactagggtccaccttttggcctctgactgaggtggaccctagtttaataaa
caggacacaaggccctttatcagcactcacatggaacaaatggccaccgtgggaggatgacaatttctgtggctgcgtgaaagccttg
aggggctccgggagctagagcctctgctaaccatgttcatgccttcttctttttcctacagctcctgggcaacgtgctggttattgtgctgt
ctcatcattttggcaaagaattcctcgaagatccgaagggaaagtcttccacgactgtgggatccgttcgaagatatcaccggttgagcc
accatgaaggttctgtgggctgccttgcttgtgacattcctggctggatgccaggccaaagtggagcaggctgtggaaacagaacctg
agcctgaactgaggcagcagacagagtggcagtctggccagagatgggaactggctttgggaaggttctgggactacctgagatgg
gtgcagaccctgtctgaacaggtgcaggaggagctgctgtctagccaagtgacccaagaactgagagccctgatggatgagaccat
gaaggagctgaaggcctacaagtctgagctggaggagcaactcacaccagtggctgaggagacaagggctaggctgagcaagga
attgcaagctgctcaggccaggttgggagcagacatggaggatgtgtgtggcagactggtgcagtacaggggagaggttcaggctat
gcttggccagagcacagaggaactgagggtgaggcttgctagccacctcaggaagctgaggaagaggctgctgagagatgctgat
gacctccagaagtgcctggctgtgtatcaagctggagctagggagggagctgaaaggggactgtctgccatcagggaaagactggg
acctctggttgagcagggaagagtgagagctgccacagtgggatctttggctggccaacctctgcaagaaagggctcaagcttgggg
agagaggcttagggccagaatggaggagatgggaagcaggacaagggacaggctggatgaggtgaaggagcaagtggctgaag
tgagggccaagctggaagaacaggcccagcagattagactgcaggctgaagccttccaagccaggctgaagagctggtttgagccc
ttggtggaggatatgcaaaggcagtgggctggcctggttgagaaagtgcaagctgctgttggcacctctgctgctcctgtgcccagtg
acaaccactgacaattgttaattaagtttaaaccctcgaggccgcaagcttatcgataatcaacctctggattacaaaatttgtgaaagatt
gactggtattcttaactatgttgctccttttacgctatgtggatacgctgctttaatgcctttgtatcatgctattgcttcccgtatggctttcattt
tctcctccttgtataaatcctggttgctgtctctttatgaggagttgtggcccgttgtcaggcaacgtggcgtggtgtgcactgtgtttgctg
acgcaacccccactggttggggcattgccaccacctgtcagctcctttccgggactttcgctttccccctccctattgccacggcggaac
tcatcgccgcctgccttgcccgctgctggacaggggctcggctgttgggcactgacaattccgtggtgttgtcggggaaatcatcgtcc
tttccttggctgctcgcctgtgttgccacctggattctgcgcgggacgtccttctgctacgtcccttcggccctcaatccagcggaccttcc
ttcccgcggcctgctgccggctctgcggcctcttccgcgtcttcgccttcgccctcagacgagtcggatctccctttgggccgcctcccc
gcatcgataccgtcgactagagctcgctgatcagcctcgactgtgccttctagttgccagccatctgttgtttgcccctcccccgtgcctt
ccttgaccctggaaggtgccactcccactgtcctttcctaataaaatgaggaaattgcatcgcattgtctgagtaggtgtcattctattctgg
ggggggggtggggcaggacagcaagggggaggattgggaagacaatagcaggcatgctggggagagatccacgataacaaac
agcttttttggggtgaacatattgactgaattcccgtgcggaccgagcggccgcaggaacccctagtgatggagttggccactccctct
ctgcgcgctcgctcgctcactgaggccgggcgaccaaaggtcgcccgacgcccgggctttgcccgggcggcctcagtgagcgag
cgagcgcgcagctgcctgcaggcatgcaagctgtagccaaccactagaactatagctagagtcctgggcgaacaaacgatgctcgc
cttccagaaaaccgaggatgcgaaccacttcatccggggtcagcaccaccggcaagcgccgcgacggccgaggtcttccgatctcc
tgaagccagggcagatccgtgcacagcaccttgccgtagaagaacagcaaggccgccaatgcctgacgatgcgtggagaccgaaa
ccttgcgctcgttcgccagccaggacagaaatgcctcgacttcgctgctgcccaaggttgccgggtgacgcacaccgtggaaacgga
tgaaggcacgaacccagttgacataagcctgttcggttcgtaaactgtaatgcaagtagcgtatgcgctcacgcaactggtccagaacc
ttgaccgaacgcagcggtggtaacggcgcagtggcggttttcatggcttgttatgactgtttttttgtacagtctatgcctcgggcatccaa
gcagcaagcgcgttacgccgtgggtcgatgtttgatgttatggagcagcaacgatgttacgcagcagcaacgatgttacgcagcagg
gcagtcgccctaaaacaaagttaggtggctcaagtatgggcatcattcgcacatgtaggctcggccctgaccaagtcaaatccatgcg
ggctgctcttgatcttttcggtcgtgagttcggagacgtagccacctactcccaacatcagccggactccgattacctcgggaacttgct
ccgtagtaagacattcatcgcgcttgctgccttcgaccaagaagcggttgttggcgctctcgcggcttacgttctgcccaggtttgagca
gccgcgtagtgagatctatatctatgatctcgcagtctccggcgagcaccggaggcagggcattgccaccgcgctcatcaatctcctc
aagcatgaggccaacgcgcttggtgcttatgtgatctacgtgcaagcagattacggtgacgatcccgcagtggctctctatacaaagtt
gggcatacgggaagaagtgatgcactttgatatcgacccaagtaccgccacctaacaattcgttcaagccgagatcggcttcccggcc
gcggagttgttcggtaaattgtcacaacgccgcgaatatagtctttaccatgcccttggccacgcccctctttaatacgacgggcaatttg
cacttcagaaaatgaagagtttgctttagccataacaaaagtccagtatgctttttcacagcataactggactgatttcagtttacaactattc
tgtctagtttaagactttattgtcatagtttagatctattttgttcagtttaagactttattgtccgcccacacccgcttacgcagggcatccattt
attactcaaccgtaaccgattttgccaggttacgcggctggtctgcggtgtgaaataccgcacagatgcgtaaggagaaaataccgcat
caggcgctcttccgcttcctcgctcactgactcgctgcgctcggtcgttcggctgcggcgagcggtatcagctcactcaaaggcggta
atacggttatccacagaatcaggggataacgcaggaaagaacatgtgagcaaaaggccagcaaaaggccaggaaccgtaaaaagg
ccgcgttgctggcgtttttccataggctccgcccccctgacgagcatcacaaaaatcgacgctcaagtcagaggtggcgaaacccga
caggactataaagataccaggcgtttccccctggaagctccctcgtgcgctctcctgttccgaccctgccgcttaccggatacctgtccg
cctttctcccttcgggaagcgtggcgctttctcaatgctcacgctgtaggtatctcagttcggtgtaggtcgttcgctccaagctgggctgt
gtgcacgaaccccccgttcagcccgaccgctgcgccttatccggtaactatcgtcttgagtccaacccggtaagacacgacttatcgcc
actggcagcagccactggtaacaggattagcagagcgaggtatgtaggcggtgctacagagttcttgaagtggtggcctaactacgg
ctacactagaaggacagtatttggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctcaagaagatcctttgatcttttcta
cggggtctgacgctcagtggaacgaaaactcacgttaagggattttggtcatgagattatcaaaaaggatcttcacctagatccttttaaa
ttaaaaatgaagttttaaatcaatctaaagtatatatgagtaaacttggtctgacagttaccaatgcttaatcagtgaggcacctatctcagc
gatctgtctatttcgttcatccatagttgcctgactccccgtcgtgtagataactacgatacgggagggcttaccatctggccccagtgctg
caatgataccgcgagacccacgctcaccggctccagatttatcagcaataaaccagccagccggaagggccgagcgcagaagtgg
tcctgcaactttatccgcctccatccagtctattaattgttgccgggaagctagagtaagtagttcgccagttaatagtttgcgcaacgttgt
tgccattgctacaggcatcgtggtgtcacgctcgtcgtttggtatggcttcattcagctccggttcccaacgatcaaggcgagttacatga
tcccccatgttgtgcaaaaaagcggttagctccttcggtcctccgatcgttgtcagaagtaagttggccgcagtgttatcactcatggttat
ggcagcactgcataattctcttactgtcatgccatccgtaagatgcttttctgtgactggtgagtactcaaccaagtcattctgagaatagtg
tatgcggcgaccgagttgctcttgcccggcgtcaatacgggataataccgcgccacatagcagaactttaaaagtgctcatcattggaa
aacgttcttcggggcgaaaactctcaaggatcttaccgctgttgagatccagttcgatgtaacccactcgtgcacccaactgatcttcag
catcttttactttcaccagcgtttctgggtgagcaaaaacaggaaggcaaaatgccgcaaaaaagggaataagggcgacacggaaat
gttgaatactcatactcttcctttttcaatattattgaagcatttatcagggttattgtctcatgagcggatacatatttgaatgtatttagaaaaa
taaacaaataggggttccgcgcacatttccccgaaaagtgccacctgaaattgtaaacgttaatattttgttaaaattcgcgttaaatttttgt
taaatcagctcattttttaaccaataggccgaaatcggcaaaatcccttataaatcaaaagaatagaccgagatagggttgagtgttgttc
cagtttggaacaagagtccactattaaagaacgtggactccaacgtcaaagggcgaaaaaccgtctatcagggcgatggcccactac
gtgaaccatcaccctaatcaagttttttggggtcgaggtgccgtaaagcactaaatcggaaccctaaagggagcccccgatttagagct
tgacggggaaagccggcgaacgtggcgagaaaggaagggaagaaagcgaaaggagcgggcgctagggcgctggcaagtgtag
cggtcacgctgcgcgtaaccaccacacccgccgcgcttaatgcgccgctacagggcgcgtc
AAV6 wild-type capsid (736 aa)
SEQ ID NO: 23
MAADGYLPDWLEDNLSEGIREWWDLKPGAPKPKANQQKQDDGRGLVLPGYKYLG
PFNGLDKGEPVNAADAAALEHDKAYDQQLKAGDNPYLRYNHADAEFQERLQEDTS
FGGNLGRAVFQAKKRVLEPFGLVEEGAKTAPGKKRPVEQSPQEPDSSSGIGKTGQQP
AKKRLNFGQTGDSESVPDPQPLGEPPATPAAVGPTTMASGGGAPMADNNEGADGV
GNASGNWHCDSTWLGDRVITTSTRTWALPTYNNHLYKQISSASTGASNDNHYFGYS
TPWGYFDFNRFHCHFSPRDWQRLINNNWGFRPKRLNFKLFNIQVKEVTTNDGVTTIA
NNLTSTVQVFSDSEYQLPYVLGSAHQGCLPPFPADVFMIPQYGYLTLNNGSQAVGRS
SFYCLEYFPSQMLRTGNNFTFSYTFEDVPFHSSYAHSQSLDRLMNPLIDQYLYYLNRT
QNQSGSAQNKDLLFSRGSPAGMSVQPKNWLPGPCYRQQRVSKTKTDNNNSNFTWT
GASKYNLNGRESIINPGTAMASHKDDKDKFFPMSGVMIFGKESAGASNTALDNVMIT
DEEEIKATNPVATERFGTVAVNLQSSSTDPATGDVHVMGALPGMVWQDRDVYLQG
PIWAKIPHTDGHFHPSPLMGGFGLKHPPPQILIKNTPVPANPPAEFSATKFASFITQYST
GQVSVEIEWELQKENSKRWNPEVQYTSNYAKSANVDFTVDNNGLYTEPRPIGTRYL
TRPL
AAV6-TM capsid (736 aa)
SEQ ID NO: 24
MAADGYLPDWLEDNLSEGIREWWDLKPGAPKPKANQQKQDDGRGLVLPGYKYLG
PFNGLDKGEPVNAADAAALEHDKAYDQQLKAGDNPYLRYNHADAEFQERLQEDTS
FGGNLGRAVFQAKKRVLEPFGLVEEGAKTAPGKKRPVEQSPQEPDSSSGIGKTGQQP
AKKRLNFGQTGDSESVPDPQPLGEPPATPAAVGPTTMASGGGAPMADNNEGADGV
GNASGNWHCDSTWLGDRVITTSTRTWALPTYNNHLYKQISSASTGASNDNHYFGYS
TPWGYFDFNRFHCHFSPRDWQRLINNNWGFRPKRLNFKLFNIQVKEVTTNDGVTTIA
NNLTSTVQVFSDSEYQLPYVLGSAHQGCLPPFPADVFMIPQYGYLTLNNGSQAVGRS
SFYCLEYFPSQMLRTGNNFTFSYTFEDVPFHSSYAHSQSLDRLMNPLIDQYLYYLNRT
QNQSGSAQNKDLLFSRGSPAGMSVQPKNWLPGPCYRQQRVSKVKTDNNNSNFTWT
GASKYNLNGRESIINPGTAMASHKDDKDKFFPMSGVMIFGKESAGASNTALDNVMIT
DEEEIKATNPVATERFGTVAVNLQSSSTDPATGDVHVMGALPGMVWQDRDVYLQG
PIWAKIPHTDGHFHPSPLMGGFGLKHPPPQILIKNTPVPANPPAEFSATKFASFITQYST
GQVSVEIEWELQKENSKRWNPEVQYTSNFAKSANVDFTVDNNGLYTEPRPIGTRFLT
RPL
AAV9 capsid (736 aa)
SEQ ID NO: 25
MAADGYLPDWLEDNLSEGIREWWALKPGAPQPKANQQHQDNARGLVLPGYKYLG
PGNGLDKGEPVNAADAAALEHDKAYDQQLKAGDNPYLKYNHADAEFQERLKEDTS
FGGNLGRAVFQAKKRLLEPLGLVEEAAKTAPGKKRPVEQSPQEPDSSAGIGKSGAQP
AKKRLNFGQTGDTESVPDPQPIGEPPAAPSGVGSLTMASGGGAPVADNNEGADGVG
SSSGNWHCDSQWLGDRVITTSTRTWALPTYNNHLYKQISNSTSGGSSNDNAYFGYST
PWGYFDFNRFHCHFSPRDWQRLINNNWGFRPKRLNFKLFNIQVKEVTDNNGVKTIA
NNLTSTVQVFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSEFAWPGA
SSWALNGRNSLMNPGPAMASHKEGEDRFFPLSGSLIFGKQGTGRDNVDADKVMITN
EEEIKTTNPVATESYGQVATNHQSAQAQAQTGWVQNQGILPGMVWQDRDVYLQGP
IWAKIPHTDGNFHPSPLMGGFGMKHPPPQILIKNTPVPADPPTAFNKDKLNSFITQYST
GQVSVEIEWELQKENSKRWNPEIQYTSNYYKSNNVEFAVNTEGVYSEPRPIGTRYLT
RNL
AAVrh10 capsid (738 aa)
SEQ ID NO: 26
MAADGYLPDWLEDNLSEGIREWWDLKPGAPKPKANQQKQDDGRGLVLPGYKYLG
PFNGLDKGEPVNAADAAALEHDKAYDQQLKAGDNPYLRYNHADAEFQERLQEDTS
FGGNLGRAVFQAKKRVLEPLGLVEEGAKTAPGKKRPVEPSPQRSPDSSTGIGKKGQQ
PAKKRLNFGQTGDSESVPDPQPIGEPPAGPSGLGSGTMAAGGGAPMADNNEGADGV
GSSSGNWHCDSTWLGDRVITTSTRTWALPTYNNHLYKQISNGTSGGSTNDNTYFGY
STPWGYFDFNRFHCHFSPRDWQRLINNNWGFRPKRLNFKLFNIQVKEVTQNEGTKTI
ANNLTSTIQVFTDSEYQLPYVLGSAHQGCLPPFPADVFMIPQYGYLTLNNGSQAVGR
SSFYCLEYFPSQMLRTGNNFEFSYQFEDVPFHSSYAHSQSLDRLMNPLIDQYLYYLSR
TQSTGGTAGTQQLLFSQAGPNNMSAQAKNWLPGPCYRQQRVSTTLSQNNNSNFAW
TGATKYHLNGRDSLVNPGVAMATHKDDEERFFPSSGVLMFGKQGAGKDNVDYSSV
MLTSEEEIKTTNPVATEQYGVVADNLQQQNAAPIVGAVNSQGALPGMVWQNRDVY
LQGPIWAKIPHTDGNFHPSPLMGGFGLKHPPPQILIKNTPVPADPPTTFSQAKLASFIT
QYSTGQVSVEIEWELQKENSKRWNPEIQYTSNYYKSTNVDFAVNTDGTYSEPRPIGT
RYLTRNL

The invention claimed is:

1. A promoter comprising a nucleotide sequence having at least about 95% sequence identity to SEQ ID NO:1.

2. The promoter of claim 1, wherein the nucleotide sequence is SEQ ID NO:1.

3. A synthetic nucleic acid comprising:
a first nucleotide sequence having at least 95% sequence identity to SEQ ID NO:1 operably linked to a second nucleotide sequence,
wherein the first nucleotide sequence is a promoter for astrocyte-directed expression, and wherein the second nucleotide sequence is not an expression control element.

4. The synthetic nucleic acid of claim 3 further comprising a third nucleotide sequence operably linked to the first nucleotide sequence, wherein the third nucleotide sequence is downstream from the second nucleotide sequence, and wherein the third nucleotide sequence is not an expression control element.

5. The synthetic nucleic acid of claim 4, wherein the second nucleotide sequence is a transgene, and wherein the third nucleotide sequence is a different transgene.

6. The synthetic nucleic acid of claim 4, wherein the second nucleotide sequence is a transgene, and wherein the third nucleotide sequence is an inhibitory nucleic acid.

7. The synthetic nucleic acid of claim 4, wherein the second nucleotide sequence is an inhibitory nucleic acid, and wherein the third nucleotide sequence is a different inhibitory nucleic acid.

8. The synthetic nucleic acid of claim 4, wherein the second nucleotide sequence is an inhibitory nucleic acid, and wherein the third nucleotide sequence is a transgene.

9. The synthetic nucleic acid of claim 5, where the transgene encodes a first Alzheimer's Disease (AD)-associated gene.

10. The synthetic nucleic acid of claim 6, wherein the inhibitory nucleic acid inhibits expression or activity of a second AD-associated gene.

11. The synthetic nucleic acid of claim 9, wherein the first AD-associated gene is APOE2.

12. The synthetic nucleic acid of claim 10, wherein the second AD-associated gene is APOE4.

13. A vector comprising the promoter of claim 1.

14. The vector of claim 13, wherein the vector is a baculovirus vector or an adeno-associated virus (AAV) vector.

15. The vector of claim 14, wherein the vector is an AAV vector, and wherein the vector further comprises a nucleotide sequence for at least one additional expression control element selected from the group consisting of an AAV ITR, an enhancer, a transcription factor binding site, an intron splice site, a post-transcriptional regulatory element, a poly A tail and a repressor binding site, and combinations thereof.

16. A recombinant adeno-associated virus (rAAV) comprising:
(i) an AAV capsid protein; and
(ii) the synthetic nucleic acid of claim 3 or the vector of claim 15.

17. The rAAV of claim 16, wherein the AAV capsid protein is an AAV6 capsid protein or variant thereof, an AAV9 capsid protein or variant thereof, or an AAVrh.10 capsid protein or a variant thereof.

18. A pharmaceutical composition comprising:
(i) the synthetic nucleic acid of claim 3, the vector of claim 15, or the rAAV of claim 16; and
(ii) a pharmaceutically acceptable carrier.

19. A method of treating an individual having or suspected of having neurodegenerative disease, the method comprising the step of:
administering to the individual an effect amount of the rAAV of claim 16.

20. The method of claim 19, wherein the administering comprises:
(i) direct injection into the central nervous system (CNS) of the individual, wherein the direct injection is selected from the group consisting of intracerebroventricular injection, intracisterna magna, intraparenchymal injection, intrathecal injection, or combinations thereof; and/or
(ii) peripheral injection, wherein the peripheral injection is intravenous injection or subcutaneous injection.

21. The method of claim 19, where the individual has Alzheimer's Disease (AD) and is homozygous for APOE4 alleles.

22. A method of expressing a nucleic acid of interest in astrocytes, the method comprising the steps of:
introducing a vector comprising a promoter comprising a SEQ ID NO:1 operably linked to the nucleic acid of interest into a cell, tissue, organ or individual.

23. The method of claim 22, wherein the nucleic acid of interest is a transgene for an Alzheimer's Disease (AD)-associated gene.

24. The method of claim 23, wherein the AD-associated gene is APOE2.

25. The method of claim 22, wherein the nucleic acid of interest is an inhibitory nucleic acid for an Alzheimer's Disease (AD)-associated gene

26. The method of claim 25, wherein the AD-associated gene is APOE4.
